(19) United States
(12) Patent Application Publication
(10) Pub. No.: US 2004/0175831 A1
(43) Pub. Date: Sep. 9, 2004

(54) Strains "fil", stress-resistant under fermentation and/or growth conditions

(75) Inventors: Johan Thevelein, Heverlee (BE); Marie-Francoise Gorwa, Lille (FR); Patrick Van Dijck, Zichem (BE); Francoise Dumortier, Heverlee (BE); Aloys Teunissen, Heverlee (BE); Katleen Lemaire, Heverlee (BE); Didier Colavizza, Conde Sur Escaut (FR); Matthias Versele, Zolder (BE); Annie Loiez, Lille (FR)

Correspondence Address: SUGHRUE MION, PLLC, 2100 PENNSYLVANIA AVENUE, N.W., SUITE 800, WASHINGTON, DC, 20037, US

(73) Assignee: LESAFFRE ET CIE

(21) Appl. No.: 10/796,166

(22) Filed: Mar. 10, 2004

Related U.S. Application Data
(63) Continuation of application No. 09/330,262, filed on Jun. 11, 1999, now abandoned.

(30) Foreign Application Priority Data
Jun. 12, 1998 (FR) 98 07463

(51) Int. Cl. C12N 15/74; C12N 1/18

(52) U.S. Cl. 435/483; 435/254.2

(57) ABSTRACT

The invention relates to new eukaryotic strains, preferably yeast strains, having the new fil phenotype, i.e. having the unexpected property of conserving good stress resistance in fermentation and/or growth phase, while conserving normal respiratory and fermentation metabolism on fermentable sugars such as glucose.

It also relates the process for obtaining such strains.

The present invention concerns new eukaryotic strains, preferably yeast strains, having the new fil phenotype, i.e. having the unexpected property of conserving or keeping good stress resistance in fermentation and/or growth phase, while conserving normal respiratory and fermentation metabolism on fermentable sugars such as glucose.

The invention also concerns a process for obtaining such strains.

The invention also concerns the use of such strains for obtaining baker's yeast with higher resistance to drying, better adapted for the preparation of frozen dough and/or for other uses where good stress resistance during the fermentation phase is required.

Yeasts of the Saccharomyces genus are used as fermentation agents in baking, brewing, winemaking, distillery and other fields. Their industrial use is based on their ability to produce carbon dioxide from sugars such as glucose, fructose, sucrose or maltose, present or added in the dough or in the wort. Fermentation ability is an important criterion of quality for yeast.

The selection of strains, the manufacturing conditions of living or active yeast have been optimized during the years so as to obtain yeast having good fermentation ability and good stress resistance under certain conditions. Unfortunately, although yeast cells harvested under conditions equivalent to those of a stationary phase have a high level of resistance to different types of stress such as heat, freezing and drying, this stress resistance is lost when a fermentation phase is initiated by adding fermentable sugars. The cells then rapidly lose their stress resistance property which causes a reduction in their fermenting power under stress conditions, this being a major disadvantage in the majority of the industrial uses for yeast.

The present invention concerns new eukaryotic strains and particularly new yeast strains obtainable by mutation (=mutagenesis), or by transformation with recombinant DNA, called “fil” strains. The use of lower-case letters indicates that they do not have the FIL phenotype which is the normal phenotype, and stands for “Fermentation-Induced Loss of stress resistance” which means loss of stress resistance induced by the fermentation. The new fil strains have a phenotype “deficient in Fermentation-Induced Loss of stress resistance” i.e. they are deficient in the loss of stress resistance which is induced by fermentation, without loss or significant alteration of their gassing power or fermentation activity and/or growth performance. In other words, the new strains conserve or keep in active metabolism phase a high level of stress resistance, comparable to that of cells which are not in fermentation or growth, i.e. are in stationary phase. This new property of the new strains is all the more unexpected insofar as the maintenance of good stress resistance is obtained without simultaneous significant loss of their growth and fermentation abilities.

All unicellular eukaryotic organisms (yeast, molds . . . ) are confronted with stress conditions whereas their industrial use requires them to be in active metabolic phase or to be able to rapidly achieve a (very) active metabolism. These two requirements, stress-resistance and active metabolism, have always been considered as contradictory and contrary to the natural biological equilibrium. For example, Attfield, in his review concerning the stress-resistance of yeasts of the Saccharomyces genus, published in December 1997 in “Nature Biotechnology”, 15, pp.1351-1357, writes that the reconciliation of these two requirements is contrary to biological design, i.e. to fundamental natural equilibrium or still to the original biological concept or definition of yeast strains. Consequently, the obtaining of new eukaryotic strains, particularly yeasts, having the unexpected properties of the fil phenotype, represents significant progress. If the example of baker's yeast is taken, it is well known that the more a cellular biomass is harvested under conditions close to exponential growth phase, the higher will be its fermenting power, but the lower will be its resistance to stress from drying or freezing. It is also well known that even if baking dough destined for freezing is seeded with a biomass harvested under conditions close to stationary phase, and consequently resistant to stress, the renewal or restarting of fermentation in the dough right from kneading and during the entire period before complete core freezing of the dough will induce a loss of this stress resistance. The new strains having the fil phenotype allow progress in the resolving of the difficulties inherent in the fundamental biological equilibrium of eukaryotic cells.

A lot of significant work has been performed in recent years to understand the mechanisms of action of stress factors on the cell and the nature of the cellular response, particularly in the case of Saccharomyces cerevisiae, when the yeast is exposed to different chemical or physical stresses. In general, the resistance appears to be a complex phenomenon, implying numerous factors which play distinctive roles. These factors, their mechanisms of action and their importance in relation to one another are still not well understood.

It is well known that cells of the yeast Saccharomyces cerevisiae become resistant to stress during stationary phase or when they are cultured or grown at a low growth rate on a non-fermentable carbon source or with a limited supply of fermentable sugar and/or nitrogen. However, this stress resistance disappears when a fermentable sugar such as glucose or maltose is supplied to the cells, which then enter rapid fermentation or active growth phase (Attfield, 1997, Nature Biotechnology, 15, pp.1351-1357; de Winde et al., 1997, Yeast Stress Responses, Ed. Springer, pp.7-52; Werner-Washburne et al., 1993, Microbiol. Rev., 57, pp.383-401).

The Ras-cAMP (Ras-cyclic Adenosine monophosphate proteins) metabolic pathway is known for its dramatic influence or role on the resistance to different types of stress in yeast cells. This has been demonstrated with respect to heat resistance (Iida, 1988, Mol. Cell. Biol. 8, pp.5555-5560; Matsumoto et al., 1985, Yeast, 1, pp.15-24; Shin et al., 1987, Mol. Cell. Biol., 7, pp.244-250), resistance to successive freezing and thawing steps (Park et al., 1997, Appl. Envir. Microbiol., 63, pp.3818-3824), and with respect to resistance to salt (Hitara et al., 1995, Mol. Gen. Genet., 249, pp.257-264).

The level of cAMP, and consequently that of protein kinase A activity in yeast cells is controlled by this elaborate and complex pathway (Broach and Deschenes, 1990, Adv. Cancer Res., 54, pp.79-139; Thevelein, 1991, Mol. Microbiol., 5, pp.1301-1307; Thevelein, 1992, Antonie Leeuwenhoek, J. Microbiology, 62, pp.109-130). In yeast, cAMP is synthesized by an enzyme, adenylate cyclase, which is encoded by the CYR1/CDC35 gene (Kataoka et al., 1985, Cell, 43, pp.493-505). The level of cAMP is, in particular, regulated and hydrolyzed by two phosphodiesterases, encoded by the genes PDE1 and PDE2 (Nikawa et al., 1997, Mol. Cell. Biol., 7, pp.3629-3636; Sass et al., 1986, Proc. Natl. Acad. Sci. USA, 83, pp.9303-9307). Furthermore, the activity of adenylate cyclase is strongly dependent on the activity of the Ras proteins (Toda et al., 1985, Cell, 40, pp.27-36).

The Ras proteins are G proteins. They are active when bound or linked to a GTP (Guanosine TriPhosphate), and inactive when bound to a GDP (Guanosine DiPhosphate). The exchange of GDP with GTP on Ras proteins is stimulated by the guanine nucleotide exchange proteins Cdc 25 and Sdc 25 (Boy-Marcotte et al., 1996, Mol. Biol. Cell, 7, pp.529-539; Camonis et al., 1986, EMBO J., 5, pp.375-380). The Ras proteins have an intrinsic GTPase activity which is stimulated by the proteins Ira1 and Ira2 and which is responsible for the downstream regulation of their activity. cAMP activates cAMP-dependent protein kinase A (protein kinase A activated by cAMP hereafter referred to as PKA), which is composed of three catalytic subunits encoded by the genes TPK1, TPK2 and TPK3 and of a regulatory subunit encoded by the gene BCY1 (Toda et al., 1987, Mol. Cell. Biol., 7, pp.1371-1327; Toda et al., 1987, Cell, 50, pp.277-287). The interaction between cAMP and the inhibiting subunit Bcy1 releases Bcy1 from the complex with the said catalytic subunits, thus activating them. These activated catalytic subunits phosphorylate a certain number of target proteins of which some have been identified, such as trehalase. The activity of PKA (protein kinase A activated by cAMP) is essential for the growth of yeast cells. When PKA activity is, in one manner or another, greatly reduced, e.g. by a severe reduction of cAMP level, the cells stop their growth and permanently enter stationary phase.

In other words, PKA, when it is activated, leads to growth of the yeast and is a mediator of different metabolic regulation processes, leading notably to a rapid decrease in trehalose content and a rapid decrease in heat shock proteins content, i.e. to disappearance of factors which favour stress resistance. On the contrary, when PKA activity is greatly reduced, the cells enter stationary phase and acquire high stress resistance. As indicated above, this has been demonstrated for different types of stress.

It is the study of these mechanisms of the complex Ras-cAMP metabolic pathway which has led Attfield review to establish that the obtaining of a phenotype corresponding to the maintenance of high stress resistance for cells in active metabolism after inoculation onto a medium containing fermentable sugars would be contrary to the “biological design” of the strains, i.e. against the natural equilibrium.

Mutants in the Ras-cAMP-protein kinase A pathway have been identified, constitutively having a high stress resistance during growth. This has been shown concerning heat resistance (Cameron et al., 1988, Cell, 53, pp.555-566; Hottiger et al., 1989, FEBS Lett., 255, pp.431-434; Shin et al., 1987, Mol. Cell. Biol., 7, pp.244-250) and freezing/thawing resistance (Park et al., 1997, Appl. Envir. Microbiol., 63, pp.3818-3824). However, these mutants have a much longer latent or lag phase at the start of fermentation and a reduced growth rate (Ma et al., 1997, Microbiol., 143, pp.3451-3459; Iida, 1988, Mol. Cell Biol., 8, page 5559), which excludes their use in industry, particularly in bakers' yeast where a rapid onset of fermentation is essential.

Certain of these mutants have other properties important for use in industry affected in a negative manner. For example, ras2 mutants are incapable of using a nonfermentable carbon source for their growth, ethanol for example (Tatchell et al., 1985, Proc. Natl. Acad. Sci., 82, pp.3785-3789 J. F. Canon and al., Genetics, 113, pp.247-264, June 1986). It is excluded to use a yeast having a deletion of the gene RAS2 as a baker's or bread-making yeast strain because the assimilation of ethanol is necessary for the growth of baker's yeasts. In other words, it seems that these mutants constitutively conserve a high stress resistance since they are incapable of entering a truly active metabolic phase.

On the contrary, deregulated mutants having a high level of cAMP or a non limited PKA activity exhibit a very weak level of both heat shock proteins and trehalose, irrespective of the culture conditions and thus also in stationary phase.

The study of these mutants which have no industrial significance confirms the conclusions of the general review by Attfield already cited above concerning stress resistance, according to which it appears unlikely to obtain industrially useful strains by classical genetics and it is thus necessary to turn to recombinant DNA technologies. However, the theoretical data allowing such an approach are insufficient for obtaining the desired new result. This is particularly illustrated by the high amount of work which was performed in the past on deletion of the gene(s) coding for a trehalase.

It is well known that in bakers' yeast a high stress resistance, e.g. to heat, to freezing or to high pressures, is correlated to an elevated trehalose content (Attfield, 1997, Nature Biotechnology, 15, pp.1351-1357; De Virgilio et al, 1994, Eur. J. Biochem., 219, pp.179-186; Iwakashi et al., 1997, Lett. Appl. Microbiol., 25, pp.43-47; Wiemken, 1990, Antonie Leeuwenhoek, J. Microbiology, 58, pp.209-217). Trehalose is a dioside present in high concentrations in a number of living organisms in nature (Van Laere, 1989, FEMS Microbiol. Rev., 63, pp.201-210; Wiemkem 1990, Antonie Leeuwenhoek, J. Microbiology, 58, pp.209-217). It possesses remarkable and apparently specific properties of protection against aggressive treatments for a whole series of biological structures (Crowe et al., 1992, Anhydrobiosos Annu. Rev. Physiol., 54, pp.579-599). Trehalose is rapidly accumulated during the phase preceding the death of yeast cells.

Initiation of fermentation by the addition of a fermentable carbon source is associated with a rapid mobilization of trehalose (van der Plaat, 1974, Biochem. Biophys. Res. Commun., 56, pp.580-587), i.e. with its metabolic degradation and its rapid disappearance. Thus, a logical approach for maintaining stress resistance during the start of fermentation has been to clone and delete the NTH1 gene, encoding for neutral trehalase (Kopp et al., 1993, J. Biol. Chem., 268, pp.4766-4774) which is the enzyme responsible for the mobilization of trehalose, so as to maintain the high trehalose level of the cells. It has been claimed that the stress resistance of yeast could be improved by the deletion of this gene (patents or patent applications EP 0451896—Hino et al., EP 0838520). However, by impeding or preventing the mobilization of trehalose by the deletion of the NTH1 gene, the rapid loss of stress resistance during the start of fermentation is not avoided (Van Dijck et al., 1995, Appl. Environ. Microbiol., 61, pp.109-115). This is also true concerning the non expression of the gene ATH1 claimed in the patent application WO 97/01626. The simple deletion of one or all the genes coding for a trehalase is not by itself capable to solve the problem subject of the present invention.

This is probably due, in particular, to the action of other resistance factors such as heat shock proteins which disappear so rapidly at the start of fermentation (Crauwels et al., 1997, Microbiol. 143, pp.2627-2637; de Winde et al., 1997, Yeast Stress Responses, Ed. Springer, pp.7-52; Praekelt et Maecock, 1990, Mol. Gen. Genet., 223, pp.97-106; Werner-Washburne et al., 1989, J. Bacteriol., 171, pp.2680-2688).

Thus, the modification of the metabolism of trehalose by genetic engineering methods has not allowed the improvement of the stress resistance of yeast and has not given practical results for the development of industrial strains resistant to stress during fermentation phase (Attfield, 1997, Nature Biotechnology, 15, pp.1351-1357).

Another approach using recombinant DNA or genetic engineering techniques has been to try to increase the stress resistance of yeast by production of antifreeze proteins present in the blood of certain fishes inhabiting very cold waters. McKown et al., (Cryobiology, 1991, 28, pp.474-482) have expressed the gene encoding for an antifreeze protein in Saccharomyces cerevisiae so as to make it produce an intracellular chimeric antifreeze protein. However, this approach has not given satisfactory results since the survival rate of the yeast after freezing is still very low.

A large number of Japanese research activities can also be cited (patent applications or patents EP 0196233—U.S. Pat. No. 4,547,374—EP 0388262) dated of the 1980-1989 decade, consisting in selecting unconventional strains for bread-making fermentation but having interesting freeze resistance properties, and crossing them with baker's yeast. This approach has yielded limited results not solving the problem subject of the invention and none of these strains was used to produce baker's yeast commercialized on the European or American market in 1998.

Thus it has not been possible until now to obtain yeast having the property of conserving a high stress resistance simultaneously with good growth and good fermenting activity.

In conclusion it can be said, in agreement particularly with the conclusions of the general review on yeast stress published in Nature Biotechnology in 1997, that:

the major problem is that the natural response of the cells, in the presence of a fermentable substrate, is to pass into active metabolic phase and thus to rapidly decrease their stress resistance factors, whereas industrial conditions necessitate active metabolism and high stress resistance

this problem was not resolved;

classic genetics have only contributed a limited improvement of the resistance of cells in industrial use conditions

a solution of this unsolved problem could only be expected via genetic engineering technologies.

Unfortunately, despite the complete knowledge of the yeast genome which has been entirely sequenced, and the knowledge which has accumulated regarding the functions of the genes but which is still very incomplete, it is only partially known how the industrially crucial properties are genetically and physiologically governed. The present state of the art is thus insufficient to allow adequate genetic manipulation to arrive at a solution of the major problem defined above. This is all the more true insofar as the regulatory pathways concerned, such as the Ras-cAMP pathway are very complex as illustrated above, and are probably numerous. The Ras-cAMP-PKA pathway is not the only metabolic (regulatory) pathway to be considered.

The present invention, in a surprising manner, has resolved these problems in a simple and efficient manner. It demonstrates that it is possible, contrary to that which was generally admitted, to obtain strains having a phenotype which was believed not to be capable of existing. This phenotype contrary to “biological design” (i.e. to natural equilibrium) has been called the fil phenotype.

### BRIEF DESCRIPTION OF THE INVENTION

The present invention concerns a process for obtaining new eukaryotic strains, preferably new yeast strains, and even more preferentially strains of Saccharomyces cerevisiae conserving stress resistance in the presence of fermentable sugars such as glucose, characterized by the fact that it comprises the following steps:

a classic mutagenic treatment is carried out on the cells of a starting or original strain,

the cells having undergone the said mutagenic treatment are cultured or grown until they reach the stationary phase,

the said cells in stationary phase are incubated in the presence of at least one fermentable sugar selected from the group comprising glucose, maltose, and sucrose, this sugar being present in a quantity such that the cells enter an active metabolic state (fermentation and/or growth) of this sugar,

said cells in active metabolic state are subjected to one or several stresses leading to a mortality rate of at least 99% with respect to the starting population,

the surviving cells are isolated and

those of the surviving cells which respond to the following criteria which characterize the fil phenotype are selected, i.e.:

a growth, evaluated by biomass production or production yield of biomass over sugar consumed in a given time or by a maximal growth rate, under identical culture conditions, at least equal to 80% of the starting or control strain, and preferably at least equal to 90% of the starting or control strain,

a CO₂ release, or a metabolite production, in identical conditions, at least equal to 80%, and preferably at least equal to 90% of the starting or control strain,

a stress resistance, corresponding to a survival rate at least 2 times higher, preferably at least 3 times higher, more preferentially at least 5 times higher, and even more preferentially at least 10 times higher than the survival rate of the starting strain, under identical phase conditions corresponding to a growth or active metabolism followed by a heat shock of at least 20 minutes at 52° C., and/or at least 1.5 times higher, preferably at least 2 times higher, more preferably at least 3 times higher, and even more preferentially at least 5 times higher than the survival rate of the starting strain, under identical conditions of growth or fermentation phase followed by freezing for a period of at least 24 hours at −20° C. or at a lower temperature,

maintenance of these properties after repeated cultures or cultivations on non selective medium, such as YPD medium, so as to verify that the fil phenotype obtained by the mutation is perfectly stable and permanent.

Preferably, the absence of all hampering properties possibly accompanying the phenotype fil will be checked. For instance, these hampering properties can be the formation of inopportune secondary metabolites, or the loss of the capacity of assimilation or fermentation of certain compounds or substrates.

According to a particular aspect of the invention, the selected fil strains preferably have the property of conserving, in growth and/or fermentation phase on fermentable sugars, at least 50%, preferably at least 60%, more preferentially at least 70%, and even more preferentially at least 80% of their survival rate with respect to the survival rate in stationary phase measured under the same conditions after a heat or freeze shock.

In a particular embodiment of the process for obtaining the new fil strains, the cells obtained after mutagenesis treatment and in stationary phase are introduced into pieces of dough of 0.5 g consisting of water (about 42.5%), flour (about 56.5%), NaCl (about 1%), at a level of 4.10⁸ cells per g of dough. Said pieces of dough are subjected to a first fermentation of 30 minutes at 30° C., then are subjected to at least 100 cycles of freezing/thawing.

Preferably, the process of obtaining the new eukaryotic strains, subject of the invention, is applied directly to industrial strains. The selection tests used correspond to the stress encountered by the said eukaryotic strain in the process(es) of production used and to the characteristics of performance of the said strain. The strain having the selected fil phenotype is a strain having the characteristics justifying its use in the industrial or craft (=artisan) production coupled with a better resistance to the encountered stresses. An industrial strain is a strain really used in optimized and competitive industrial production. If the starting (=original) strain is a baker's yeast strain, this strain is then a strain actually used by a specialized yeast producer for the selling on the market of baker's yeasts, or a strain having equivalent properties.

A laboratory strain is a model strain allowing a good understanding of the studied phenomena, but which does not have all the properties necessary to industrial strains. A yeast model strain is a true haploid or a true diploid of Saccharomyces cerevisiae, which can contain a certain number of auxotrophic markers. The industrial yeast strains are usually polyploids.

In a particular embodiment of the process for obtaining new fil strains, wherein an industrial fil mutant strain carrying several mutations, then one can improve the said mutant in the following manner: Process according to claim 3, wherein an industrial fil mutant carrying several mutations is obtained and wherein:

the segregants issued from this industrial mutant are crossed with a laboratory haploid strain to select the segregant issued from this industrial mutant giving to the polyploids obtained with the laboratory strain an improvement in the required properties;

the segregants thus selected are crossed one with the other;

the polyploids obtained are selected according to the above-defined criteria of fil phenotypes.

The present invention also concerns new eukaryotic strains, preferably new yeast strains belonging to the Saccharomyces genus, preferably Saccharomyces cerevisiae, having the fil phenotype, obtainable by the process described above or one of its embodiments.

The present invention concerns:

on the one hand new laboratory yeast strains, new haploids or segregants or segregeants of laboratory strains or industrial strains having the fil phenotype, these new strains being essentially tool or model strains for the construct of industrial strains possessing the fil phenotype

on the other hand, and this is the main subject of the invention new industrial yeast strains, preferably new industrial baker's yeast strains possessing the fil phenotype.

An advantageous particularity of the present invention is to obtain industrial eukaryotic fil strains not GMO, that is not Genetically Modified microOrganisms in the meaning of the directive CEE 90/220 for example. Considering the unjustified European reluctance, in the light of the precise regulation existing in Europe towards GMOs, it is significant that the process subject of the invention allows to construct new industrial baker's yeast strains that possess the fil phenotype and that are not genetically modified, i.e. that are non-GMO.

In particular, these new yeast strains presenting the phenotype fil preferably have a survival rate, in growth phase on fermentable sugar, of at least 50%, preferably at least 60%, more preferably at least 70%, and even more preferably at least 75%, after heat treatment of 20 minutes at 52° C., the growth phase being defined:

for laboratory strains (true haploid or diploid strains, generally auxotrophic) and all segregants of industrial strains, as a reculturing on fermentable sugar (glucose) for 30 minutes at 30° C. after stationary phase, i.e. as a cultivation of stationary cells for 30 minutes at 30° C.;

for industrial strains (aneuploid and polyploid strains), as a cultivation of stationary cells on fermentable sugar (glucose) for 10 minutes at 30° C. after stationary phase.

The new yeast strains according to the invention are all new yeast strains of the laboratory type (true haploid or diploid strains, generally auxotrophic) and all segregants of industrial strains, whose stability to freezing in pieces of dough containing 20 g of flour, 15 g of water, 1 g of sucrose, 0.405 g of NaCl, 0.06 g of (NH₂)₂SO₄ and 160 mg of dry matter of the considered strain is at least higher than 60%, preferably at least higher than 70% and more preferably at least higher than 80%, stability being defined by the ratio between the release of CO₂ at 30° C. after 1 month (30 days) of conservation at −20° C. and the release of CO₂ at 30° C. after 1 day of conservation at −20° C.

The new yeast strains according to the invention are also all new industrial yeast strains (aneuploid or polyploid strains), whose stability to freezing in pieces of dough containing 20 g of flour, 15 g of water, 0.405 g of NaCl, 0.06 g of (NH₄)₂SO₄ and 160 mg of dry matter of the considered strain, measured by the ratio between the release of CO₂ at 30° C. after 1 month (30 days) of conservation at −20° C. and the release of CO₂ at 30° C. after 1 day of conservation at −20° C., is at least higher than 80%, preferably at least higher than 85% and more preferably at least higher than 90%.

These freeze-stability tests- correspond respectively to tests C2 and C1 described hereafter. Before freezing at −20° C., these pieces of dough are incubated at 30° C. for 30 minutes (test C1 on industrial yeast) or 60 minutes (test C2 on laboratory strains or segregants).

Preferably, the new yeast strains according to the invention allow the obtaining of dry yeast from a biomass harvested in exponential growth phase or in phase close to exponential growth phase, having a loss or decrease of released gas (=gassing power) after drying at most equal to 67%, preferably at most equal to 50% of the loss of released gas after drying of yeasts obtained using the corresponding starting or original strain (non mutated) or a control strain having the same characteristics.

The present invention also concerns the new strains having the fil phenotype:

PVD1150=M5 fil1 deposited at C.N.C.M. 25 rue du Docteur Roux, F-75724 PARIS cedex, under the n° I−2031 (contaminated strain) and I-2203, in accordance with the Budapest Treaty.

KL1=W303 fil2 deposited at C.N.C.M. 25 rue du Docteur Roux, F-75724 PARIS cedex, under the n° I−2032, in accordance with the Budapest Treaty.

FD51=HL816 fil300 deposited at C.N.C.M. 25 rue du Docteur Roux, F-75724 PARIS cedex, under the n° I−2033, in accordance with the Budapest Treaty.

FDH16-22=HL822 fil300 deposited at C.N.C.M. 25 rue du Docteur Roux, F-75724 PARIS cedex, under the n° I−2034, in accordance with the Budapest Treaty.

AT25=S47 fil400 deposited at C.N.C.M. 25 rue du Docteur Roux, F-75724 PARIS cedex, under the n° I−2035, in accordance with the Budapest Treaty.

AT28=S47 fil500 deposited at C.N.C.M. 25 rue du Docteur Roux, F-75724 PARIS cedex, under the n° I−2036, in accordance with the Budapest Treaty.

and all the new strains of the same kind, that is to say all the new strains having similar characteristics.

As the first deposit of the strain PVD1150=M5 fil1 revealed to be contaminated, it was deposited again on May 20th, 1999 under the number I-2203.

The invention also concerns a mutant or mutated gene or mutated genes which confer the phenotype fil, this or these genes being obtained by molecular biology techniques from eukaryotic fil strains obtained by the process of obtaining fil mutants. In particular, the said gene is the gene or genes which confer(s) the phenotype fil to one of the fil strains which exemplify the present invention and which have been deposited at the C.N.C.M. For example, the said gene is the gene CDC35=CYR1 carrying a mutation conferring the fil phenotype.

Advantageously, the said mutation in the gene CDC35=CYR1 is a change of a G base (guanine) into an A base (adenine) in the region of the gene CDC35/CYR1 coding for the catalytic site of the enzyme, equivalent to a change of an acidic amino acid (glutamic acid) into a basic amino acid (lysine) at position 1682 of the protein. This mutation is responsible for the fil phenotype in the strain PVD1150=M5 fil1.

This gene can also be the gene YDL 035C according to the nomenclature defined in the publication concerning the sequencing of the yeast genome, project published in Nature, 1992, 357, pp.38-44, hereafter called GPR1, carrying a mutation conferring the fil phenotype and more particularly the mutation of the strain KL1=W303 fil2. This gene can also be the mutated gene or one of the mutated genes conferring the fil phenotype of one of the strains C.N.C.M. I-2033, I-2035, I-2036.

In a general manner, the present invention includes all genes encoding for a protein having similar or equivalent properties to the proteins encoded by the genes defined above as carrying a mutation conferring the fil phenotype, i.e. all genes carrying a mutation conferring the fil phenotype and belonging to the family of genes:

encoding for a protein having a function comparable or equivalent to that of a protein encoded by one of the genes defined above as carrying a mutation conferring the fil phenotype in yeast or another eukaryote, a comparable function being defined as the commanding of the same mechanisms in the same metabolic pathway or an equivalent metabolic pathway,

encoding for proteins associated with the protein encoded by one of the genes carrying a mutation conferring the fil phenotype,

encoding for proteins having similar sequences, i.e. at least 60% homology or identity, preferably at least 70% homology and still more preferably at least 80% homology with the protein encoded by one of the genes carrying a mutation conferring the fil phenotype,

For example, this gene can encode for a protein associated with the protein encoded for by the gene GPR1 defined above, and specifically, this gene can be the gene(s) GPA2 of yeast carrying a mutation which confers the fil phenotype.

In general, the present invention is not limited to yeast strains and concerns all eukaryotic strains carrying a fil mutation i.e. having a fil phenotype.

Said eukaryotic strain is advantageously transformed in a manner so that at least certain of the alleles of a gene, capable once mutated of conferring the fil phenotype, carry the mutation conferring the said fil phenotype. Said eukaryotic strain is preferably an industrial strain and preferably an industrial yeast strain.

The invention also concerns the use of yeast strains mutated and selected for their fil phenotype obtainable by the process, subject of the invention, or the use of strains genetically transformed in such a manner as to have the fil phenotype, for obtaining bread making yeast (=baker's yeast), in particular destined to the inoculation of frozen dough.

The present invention also concerns the use of the said mutated or transformed fil strains for obtaining dry bread making (=baker's) yeast.

The present invention also concerns the use of said mutated and selected or transformed fil strains for obtaining industrial brewer's yeast, winemaking yeast or yeasts destined for the production of alcohol.

In a general fashion, the present invention concerns the use of new eukaryotic strains having the fil phenotype in any industrial condition necessitating simultaneously properties of resistance to stress and of active metabolism of said eukaryotic strains.

### DETAILED DESCRIPTION

The invention concerns new eukaryotic mutant strains called fil, preferably new fil yeast strains, which conserve or keep high stress resistance during an active growth or active fermentation phase on glucose and which have conserved the essential part of their metabolic properties (growth, production of primary or secondary metabolites). These characteristics of fil strains were considered as irreconcilable or incompatible and contrary to biological design. The search for such new strains consequently went against a preconceived opinion. The new process used for the obtaining of such eukaryotic strains having unexpected properties is based on the following steps:

the cells are subjected to a known mutagenesis treatment or protocol;

the cells thus obtained are cultured or grown until they reach stationary phase, then are recultured or cultivated in the presence of a fermentable sugar such as glucose to be in active metabolism;

said cells are subjected to a strong heat or freeze stress, i.e. a stress causing high lethality, so as to select those which have become resistant to stress in active metabolic phase;

the different successive steps described above or at least one of them may be repeated so as to obtain a survival rate with respect to the starting population equal to or lower than 1% and preferably to 1 per 1000, or even 1 for 1 million;

the surviving mutated or mutant strains (cells) thus obtained are tested to verify their resistance to heat or freezing in growth or fermentation phase, preferably they are also tested to check the non appearance of an undesired secondary property or the non disappearance of an interesting or useful secondary property, then are selected from among the resistant strains those strains which have substantially conserved their properties of growth, fermentation and/or synthesis of metabolites of industrial interest, preferably without appearance of hampering properties and without disappearance of useful properties;

the maintenance of properties corresponding to the fil phenotype is finally verified after cultivation of the said strain corresponding to a large number of generations under non selective conditions for cells.

This process of searching for fil strains may be applied to all eukaryotic strains or organisms, the tests optionally being adapted as a function of the characteristics of the said eukaryotic strain. It is noted that due to the fact that the resistances are generally crossed, the tests of resistance to heat and/or freezing are good selection tests for the search for any new strain resistant to a given stress that it will encounter during its industrial use. In order to simplify the language, these two tests are referred to below as tests of resistance to thermal shock.

In an entirely unexpected manner, since it was thought that such strains did not exist, the process thus described effectively led to the selection of several perfectly stable strains having these characteristics corresponding to the fil phenotype and notably to the selection of new industrial baker's (or bread-making) yeast strains, not GMO and directly usable to the commercialization of new baker's yeast strains.

The culture media used in the present invention are:

| | | | |
|---|---|---|---|
| YPD | yeast extract | 10 | g/l |
| medium | bactopeptone | 20 | g/l |
| | glucose | 20 | g/l |
| YPD-A | yeast extract | 10 | g/l |
| medium | bactopeptone | 20 | g/l |
| | glucose | 20 | g/l |
| | agar | 20 | g/l |
| YP | yeast extract | 10 | g/l |
| medium | bactopeptone | 20 | g/l |
| SD-URA | Nitrogenous base | 6.7 | g/l |
| | medium free of | | |
| | aminoacid | | |
| | (Yeast Nitrogen | | |
| | Base DIFCO ®) | | |
| | complementary | 0.77 | g/l |
| | mixture without | | |
| | uracil (CSM-URA, | | |
| | Bio 101 ®) | | |
| | glucose | 20 | g/l |
| | agar | 15 | g/l |
| R | glucose | 1.5 | g/l |
| medium | yeast extract | 1.0 | g/l |
| | MgSO₄ | 0.7 | g/l |
| | CaCl₂ | 0.4 | g/l |
| | (NH₄)₂SO₄ | 2.0 | g/l |
| | KH₂PO₄ | 1.87 | g/l |
| | K₂HPO₄ | 1.1 | g/l |

The tests used in the present invention are:

### Tests for Screening of Mutants Resistant to Thermal Stress During Active Fermentation and/or Growth Phase

Test T1: Survival Rate of Cells after Heat Thermal Shock

The cells are cultured or grown under stirring on YPD medium until stationary phase is obtained. These cells in stationary phase are washed, resuspended in ice-cold YP medium, so that the optical density at 600 nm is preferably comprised between 1 and 2 with respect to the medium. This suspension is then incubated at 30° C. until said temperature is reached. Part of the suspension is then kept on ice in order to serve as a control for stationary phase cells. Glucose is added to the other part to a final concentration of 100 mM. This addition is followed by an incubation of cells suspension at 30° C. of 10 to 90 minutes and this other suspension part is then kept on ice at the end of the chosen time. Both suspensions are subjected, on the one hand, to a counting of the number of viable cells after suitable dilution and, on the other hand, to a thermal treatment of at least 20 minutes at 52° C. followed by the same counting of the number of viable cells. This counting is performed on YPD-A after two day incubation at 30° C.

A measurement of the stress resistance or survival rate on the one hand of cells in stationary phase and on the other hand of cells in active metabolism can thus be obtained. The survival rate is expressed as a ratio of the number of colonies formed in the thermally treated samples and the number of colonies formed in the control samples.

Test T2: Survival Rate of Cells after Cold Thermal Shock

Identical to Test T1 except for the stress applied: the suspension is incubated at −20° C. to −30° C. for 1 to 12 days.

Test T3: Determination of the Growth Rate

The cells are cultured on YPD medium at 30° C. under stirring at 180 rpm until stationary phase is obtained.

The growth, i.e. the proliferation of cells is monitored as a function of time by measurement of the absorbency (=optical density) at 600 nm with respect to non seeded medium.

It is expressed by the curve of development of the absorbency, i.e. by the change of optical density in function of time.

The growth rate is the rate of increase of the number of cells, i.e. the slope of the absorbency curve as a function of time.

Test T4: Determination of Growth Rate in Microplates

The cells are cultured on YP medium+glucose at 10 or 100 mM concentrations or on YP medium+beet molasses at 5 g/l at 30° C. and in microtitration plates (microplates). 250 μl of medium are seeded in order to obtain 0.05 OD at 600 nm of cells in stationary phase. The microplates are shaken for 30 seconds of each minute and the absorbency at 600 nm is measured every 30 seconds. The growth rate corresponds to the slope of the absorbency curve, the μmax or maximum growth rate is then determined.

Test T5: Production on Molasses Medium in a Given Time

Dishes containing 100 grams of agar medium having the following composition:

| beet molasses | 5 | g/l |
|---|---|---|
| (NH₄)₂HPO₄ | 0.5 | g/l |
| agar | 26 | g/l |
| pH | 5-5.5 | |
| biotin added | 0.5 | μg/l |
| after | | |
| autoclaving | | |

are seeded or inoculated with the equivalent of 2 mg dry matter of yeast per dish. These dishes are incubated at 30° C. for 20 to 40 hours. The final quantity of yeast dry matter produced in weight is measured in 10 to 20 dishes, generally 16 dishes.

For all strains having an auxotrophy, the medium is complemented with the corresponding nutrient.

Tests T6 and T7 Measuring the Fermentative Capacity (T6) and the Loss of Fermentative Capacity after Freezing (T7)

To test the loss of the fermentative capacity, the glucose consumption of frozen cells is measured and compared the glucose consumption of the same non frozen cells but stored on ice. The residual fermenting power after freezing is then determined by the ratio between the glucose consumption of frozen cells and the glucose consumption of non frozen cells. This ratio corresponds to test T7, the glucose consumption of non frozen cells corresponds to test T6.

Cells are first incubated at 30° C. on YPD medium until the obtaining of the stationary phase. They are resuspended in fresh YP medium and incubated at 30° C. for 30 minutes. Glucose is then added to a final concentration of 100 mM and the incubation continues for another 30 minutes without agitation and ventilation, so that to be in fermentation conditions.

Afterwards, cells are resuspended in YP medium to obtain an Optical Density (OD) at 600 nm equal to 15. Samples of 0.03 ml are removed and frozen directly in a methanol bath of −25° C. during 1 hour, then are stored at −30° C. during 1 to 36 days then thawed at ambient temperature.

In parallel, samples are stored on ice and are used as unfrozen control cells. Thawed cells samples and control cells samples are 11 times diluted with YP-10 mM glucose and incubated at 30° C. 0.01 ml cell free samples are collected after 90 and 120 minutes. Residual glucose is measured by adding 0.2 ml Trinder Reagent® (supplier: Sigma). After 15 minutes of incubation at 30° C., the absorbance is measured at 505 nm. An almost linear correlation is found between the amount of active cells and the glucose consumption making it relatively simple to determine the residual fermentation capacity of the frozen samples.

### Tests for Determining the Gassing Power of Yeast

Tests A1, A20, A′1, A′20, C1 and C2, used for determining fermenting or gassing power of yeast, i.e. their capacity to produce CO₂, are performed with the help of a fermentometer from Burrows and Harrison, described in “Journal of the Institute of Brewing”, 1959, LXV, 1, January-February, and are precisely defined in the following manner:

Test A1: Fermenting Power (Fresh Yeasts, Industrial Strains)

To 20 g of flour incubated at 30° C. is added a weight of yeast corresponding to 160 mg dry matter, this yeast being suspended in 15 ml of water containing 27 g of NaCl per liter and 4 g of (NH₄)₂SO₄ per liter; the suspension is mixed with a spatula for 40 seconds, so as to obtain a dough which is placed at 30° C.; thirteen minutes after the beginning of mixing, the recipient (pot) containing the dough is hermetically sealed; the total quantity of carbon dioxide (CO₂) produced is measured after 60 and 120 minutes, this quantity is expressed in ml at 20° C. and under 760 mm of mercury (Hg).

Test A20: Fermenting Power (Fresh Yeasts, Other Strains)

Test identical to test A1 but the composition of the dough is modified as follows: 1 g (1 gram) of sucrose is added to the mixture of flour, water, yeast and salt, before carrying out the kneading. Furthermore, the release of CO₂ is measured after 120 and 240 minutes at 30° C. (instead of 60 and 120 minutes for test A1).

Test A′1: Fermenting Power (Dry Yeasts, Industrial Strains)

Test identical to test A1 but before mixing, the 160 mg of dry matter content of yeast, which has the form of dry yeast, is rehydrated for 15 minutes in distilled water, at 20° C. or 38° C.; 40% of the volume of water used for the rehydration is used to this effect; the complement in water, with the addition of 405 mg of NaCl is added after the 15 minutes of rehydration.

Test A′20: Fermenting Power (Dry Yeasts, Other Strains)

Test identical to test A′1, but 1 g of sucrose is added to the flour; the total quantity of gas produced is measured over 240 minutes.

Test C1: Fermenting Power (Industrial Strains after Freezing)

Test identical to test A1 but it is necessary to prepare at least six pieces of dough per strain. The pieces of dough are prepared or made according to the conditions of test A1, but after kneading, the pieces are incubated at 30° C. for 30 minutes. The prefermented pieces of dough are then immediately stored at −20° C. and conserved at this temperature for periods from 1 day to 2 months. To measure the fermenting activity after conservation at −20° C., the frozen dough is placed in an incubator at 30° C.; after thirteen minutes, the pot containing the dough is hermetically sealed and the total quantity of CO₂ produced (expressed in ml at 20° C. and under 760 mm Hg) is measured after 120 minutes with a fermentometer of Burrows and Harrison.

For a given strain, the reference release of CO₂ corresponds to the release of CO₂ of a dough sample stored at −20° C. for a single day. The other pieces of dough are thawed at regular intervals (for example after 1 week, 2 weeks, 1 month (i.e. about 30 days), 1½ months and 2 months of storage at −20° C.), and the release of CO₂ is measured so as to follow the evolution of the fermenting activity as a function of the duration of storage at −20° C.

The stability to freezing is defined as the ratio of CO₂ release during 2 hours after 1 month of freezing and the reference CO₂ release during 2 hours after 1 day of freezing.

Test C2 (Other Strains after Freezing)

Test based on test C1 with the following modifications:

1 g of sucrose is added to the flour-water-salt-yeast mixture before kneading,

the pieces of dough are prefermented 60 minutes at 30° C. before freezing at −20° C.,

the CO₂ release is measured during 240 minutes at 30° C.

Test R (Determination of Ethanol Assimilation)

100 ml of R medium are inoculated with stationary phase cells, previously grown on YP medium+glucose at 100 mM during 24 to 48 hours, so that the optical density (OD) of the inoculated culture is 0.05 OD at 600 nm. The inoculated R medium is then incubated at 30° C. and stirred continuously at 180 rpm (revs per minute). The absorbency (optical density) at 600 nm is monitored with regular takings until obtaining a stable OD. Within these conditions, a strain, which does not assimilate ethanol reaches an OD about 4 times lower than a strain normally assimilating ethanol. Only the strains reaching an OD at least equal to 50% of the control strain (in general, the non mutated strain) are retained, and preferably at least 80%, and more preferably at least 90%.

All the tests described above for putting into practice of the invention are tests of a biological nature and their reproducibility from one laboratory to another often causes problems of a delicate nature. Consequently, they should usually be interpreted in a relative fashion with respect to a control. The tests should be conducted so as to reproduce the values indicated for the control, or preferably, the controls so as to have a scale of reproducible values. This conducting of the tests should be carefully and meticulously performed.

It is clear for the person skilled in the art that each step of the process should be adapted in accordance with the characteristics of the strains used and their lethality or survival rate in the different tests. The indications given hereafter concerning the carrying out of the different steps of the process, subject of the invention, are only examples for the application of the process to Saccharomyces cerevisiae.

The process object of the invention whose principle is given hereabove and which is intended to find new strains that simultaneously have an active metabolism and are stress resistant, may be carried out:

on lab strains, i.e. on model strains whose genetic characteristics are well known and which generally contain auxotrophy markers;

on industrial strains, in general much more complex on the genetic level, due to the fact that they are not true haploids or diploids, contrary to lab strains, said industrial strains having been selected on the basis of their industrial performances. Among the industrial strains, it is possible to distinguish:

the industrial strains in their most stable state, in general in polyploid form,

the segregants of industrial strains, i.e. the sexual forms which are in general less stable, but which can be used for the constructions carried out by classic genetics.

Within the frame of the research carried out in order to characterize the mutations providing the fil phenotype, the new process object of the invention will be preferably carried out on lab strains, preferably haploids.

The characterization of the gene or of the genes involved will be carried out to allow subsequent constructions of industrial strains, carrying the corresponding mutation or mutations.

Within the frame of research carried out in view of obtaining directly industrial strains, the process will be carried out directly either on segregants of industrial strains or directly on industrial strains. The use of segregants means that the fil segregants thus obtained will be subjected to constructions by classic genetics in order to recover all desired characteristic features of industrial strains, in addition to the fil phenotype.

The obtaining of fil industrial strains, not GMO, which means not Genetically Modified Organisms, is the preferred subject of the invention. The main characteristics of these industrial strains are the following ones:

preservation of the main properties of the industrial strains actually used because of their efficiency (performance) they are derived from

significantly increased resistance to the stress conditions met in the processes of industrial or craft production they are used in

contribution to a progress (like best productivity or lower industrial cost) or a best result linked to the fil phenotype.

According to its intrinsic definition, the fil phenotype cannot correspond for a yeast strain for example:

to a deletion of RAS1 or RAS2 genes, considering the poor assimilation of ethanol such a deletion implies (J. F. Cannon and al., Genetics, 113, p.250, June 1986). The fil strains must demonstrate that they are able to re-assimilate one part of the alcohol in the test R because this is a useful secondary property which must not disappear.

to the cyr1-T1 and cyr2-T2 mutants described by Iida (Mol. Cell Biol., Dec. 1988, pp.5555-5560) considering the low growth rates of these mutants.

to a particular sensitiveness of its multiplication or its fermentation to temperature.

to a simple deletion of one or several genes encoding for one of the trehalases of yeast, the non synthesis or the low synthesis of trehalases in growth condition being an interesting factor as a secondary factor of the stress resistance, but insufficient by itself.

to a transformation with DNA encoding for a protein with a SOD activity (SuperOxide Dismutase) and with DNA encoding for a protein with catalase activity, because this corresponds to secondary factors with limited action, and to loss of efficiency or performance linked to the expression of these genes within the scope of the normal use of such yeasts.

A bread-making yeast strain, which after mutation, would give for instance a bad smell to breads, a bad or abnormal taste to breads, due to a secondary metabolism affected by the mutation, would not be a strain corresponding to the fil phenotype, because according to its preferential definition given in the present invention, the fil phenotype corresponds to a yeast having not any secondary property undesirable for its use.

Advantageously, the mutagenic treatments used in the process according to the invention are as follows. The cells of Saccharomyces cerevisiae yeast are cultured on YPD medium and are then subjected to a mutagenic treatment using a chemical agent such as EMS (Ethyl-Methyl-Sulfonate) or by ultraviolet light according to the classic protocols (Sherman et al., 1986, Cold Spring Harbor Lab Press; Spencer et al., 1988, Yeast a practical approach, Ed. Campbell et Duffus). The conditions of the mutagenesis are in general selected in order to obtain a survival rate of the cells of the order of 1 to 20%, preferably about 10%. The cells are then washed, suspended again in YPD medium and cultured until the stationary phase is obtained. Then a known amount of the culture is taken, possibly washed, transferred on YPD medium and incubated at 30° C., for 30 to 90 minutes. According to a variant, it is possible to add to the culture in stationary phase 100 mM glucose and to incubate at 30° C. for 30 to 90 minutes.

The culture is then subjected either to a heat shock by incubation between 52 and 65° C., preferably at 56° C. for 30 minutes or more, or to a cold shock (freezing) by incubation between −20° C. and −40° C. for 1 to 3 days. The freezing induces only a small loss of viability of the yeast cells, the treatment is repeated up to 200 times, until a survival rate of the cells lower than 1 for 10000, preferably lower than 1 for 100000 and still more preferably lower than 1 for 1000000 is obtained.

In a particular embodiment of the selection protocol, the cells after mutagenesis are introduced in small pieces of dough of about 0.5 g in a proportion of 4.10⁸ cells/g of dough, the latter being composed of water (42.5%), of flour (56.5%), of NaCl (1%) and subjected to fermentation for 30 minutes at 30° C. in such a way that the yeast cells leave the stationary phase, i.e. enter the fermentation phase. The pieces of dough are then successively frozen at −30° C. and thawed at room temperature, up to 200 times in such a way that only a few hundred or a few thousand cells survive.

At this stage, the cells, which survived the thermic shock (heat or freezing) are mutants, which are stress resistant and of which it is necessary to check, on the one hand, the persistence of the said property during fermentation or growth for several generations and, on the other hand, the maintenance of their growth ability and that of the production of metabolites.

In order to check the stress resistance in growth phase and/or in fermentation phase, the survival rates are measured according to the tests T1 and/or T2, and/or equivalent tests. The mutated strains are selected according to the increase of their survival rate under an important stress during or after fermentation phase and/or growth phase like those according to tests T1 and T2 in comparison with the starting strain, and in that scope, from a general point of view, a survival rate of at least 1.5 times higher will be required. The mutated strains can also be selected by comparison of their survival rate in growth phase and/or fermentation phase with respect to their survival rate in stationary phase, or with respect to the survival rate in stationary phase of the starting (original, non modified) strain.

Preferably, the selected mutated strains will present, after a growth and/or fermentation phase of the kind defined in tests T1 or T2 at least 50%, preferably at least 60%, more preferably at least 70%, and still more preferably at least 80% of their survival rate in stationary phase.

According to a variant, the resistance of the mutated strains which are obtained is measured, after freezing according to the tests C1 or C2 or according to equivalent tests, or still again the resistance against drying is measured using the ratio starting from the values obtained in tests A and A′ with respect to the yeast before and after drying.

In order to check the conservation of the growth and fermentation properties, a comparison can be made between the starting strain and the mutated strain having a phenotype of stress resistance in metabolic active phase, in one of the tests T3, T4 or T5 and in one of the tests A or in similar tests which permit the measurement of the properties which are of interest for the considered strains. A conservation of at least 80% of these properties is necessary to correspond to the fil phenotype.

Preferably, it will be checked that the mutation did not lead to any loss of an interesting secondary characteristic. For example, it will be checked by test R that the strain has a sufficient alcohol assimilation, at least 50% of that of the control strain. It will also be checked that there is no production of inopportune metabolite. For example, it will be checked on the industrial baker's yeast strains that under the usual conditions of bread-making notably in Europe and in the USA, there is no bad taste or bad smell given to-the breads.

Furthermore, it is necessary to check that the mutation is stable, in other words that the fil phenotype is stable. Consequently, all the above-defined verifications should be repeated on the reisolated mutated strain after a great number of multiplications in non selective medium, as for example after 10 successive cultures of 2 days in YPD-A medium at 30° C. or again after 10 cultures in YPD medium at 30° C., each culture being seeded by 1 per cent from the preceding culture.

The strains which have withstood with success these different selection screenings are strains which are perfectly stable and which present the fil phenotype according to the invention. They are resistant to the stress(es) in active metabolism phase and they have still an interesting growth and fermentative power.

The fil strains are intended to be used in industrial applications in which the stress resistance during the active metabolism phase must be high. The properties intended to be obtained for the fil strains are for example a high stability during freezing and/or during drying. The word stability denotes that the fermentative power of the yeast which has been placed under agressive conditions maintains a high level with respect to the fermentative or gassing power of the same yeast before the aggressive treatment, especially the drying and/or the freezing within a piece of dough.

The stability of the yeast activity of frozen dough after thawing is an important criterion in industrial bakery where the pieces of dough are frozen after beginning of the fermentation, the duration of freezing being from a few days to a few weeks. It is important that the yeast when thawed has not lost the essential part of its fermentative power.

The industrial yeast strain which, on the one hand, has properties which are equivalent to those of industrial strains which are at present marketed in France or in Europe and which, on the other hand, has a stability during freezing in pieces of dough according to test C1 at least higher than 80%, preferably at least higher than 85% and still preferably at least higher than 90%, represents an important progress for the making of frozen doughs. Preferably, this industrial baker's yeast strain is not GMO.

From the point of view of the selection of fil mutants starting from lab strains in order to characterize subsequently the gene(s) which are at the origin of the mutation, or of segregants which are intended to be used for the construction of new industrial strains by classic genetics, experience has shown that it was necessary to adopt at the level of the selection the stability rates which are lower as far as frozen dough is concerned, these strains being in general naturally less resistant to freezing stress.

The baker's yeast, in dried form, presenting at least 92% of dry matter, preferably at least 94% of dry matter, should maintain its fermentative performances, calculated for the same dry matter amount, despite the stress due to dehydration.

It is well known that yeasts present a loss of activity after drying which is all the more lower as they have been harvested during a phase of low growth, that is to say under conditions which are relatively far from those at which they present their maximum fermentative potential. Fil strains allow the shifting of this equilibrium. That possibility can be measured by comparison of a loss of activity after drying of the fil strain with respect to the loss of activity after drying of the starting strain or of a control strain cultured under the same conditions of active growth phase. The loss of activity after drying is defined according to the formula:

$$100 - \frac{A '}{A} \times 100$$

A′ represents the fermentative power measured on the dry yeast according to test A′, A is the gassing power measured for the yeast before drying according to the corresponding A test.

The process according to the invention has led to the obtaining of two types of mutants of yeast laboratory strains called fil1 and fil2, a different numerical significance (fil1, fil2, . . . ) being given for each mutation appearing to affect a different gene.

The first family of mutants is the fil1 family of which the typical strain is the strain PVD1150=M5 fil1. This mutant is derived from the wild type laboratory strain M5 (Schaaff et al., 1989, Curr. Genet., 15, pp.75-81). The fil1 mutation is carried by the gene CYR1/CDC35, which encodes for adenylate cyclase, that is for the enzyme which synthesizes cAMP. The mutation was identified as being a substitution of a glutamate residue by a lysine residue in position 1682 of the protein. This exceptional change is located in the catalytic domain of adenylate cyclase, close to the region which is considered as being implicated in the activation of adenylate cyclase by the Ras proteins. The change of an acidic aminoacid into a basic aminoacid is capable of strongly influencing the activity of the catalytic site, all the more since the change occurs in a very highly conserved zone. This fil1 mutation introduced in the gene CYR1/CDC35 of 2 different laboratory strains, caused them to acquire the said fil phenotype. Consequently, the fil1 mutation in the gene CYR1/CDC35 allows the construction of industrial strains having the fil phenotype.

The second family of mutants is the fil2 family of which the typical strain is the strain KL1=W303 fil2. This mutant is derived from the laboratory strain W303-1A (Thomas et Rothstein (1989) Cell, 56, pp.619-630).

The fil2 mutation is carried by the gene YDL035c called gene GPR1 by Xue et al, EMBO J., 1998, 17, 1996-2007. The gene GPR1 was isolated and identified as being a gene coding for a protein associated with the Gpa2 protein coded by the gene GPA2. The introduction of this mutated GPR1 gene in industrial strains will allow to confer the fil phenotype to these strains.

Mutations of at least a gene belonging to the family of genes coding for proteins associated with the protein coded by the gene GPR1, as a mutation on the gene GPA2, are capable with a very high probability to lead to the fil phenotype.

The process according to the invention has led to obtain two industrial segregants carrying the mutation called fil300.

These are the strains FD51=HL816 fil300 and FDH16-22=HL822 fil300.

These two strains are derived from an industrial segregant. They allow by crossing with other industrial segregants, then selection of at least two segregants of different mating type carrying the fil phenotype, and finally crossing between these segregants, to construct new industrial strains having the fil phenotype.

Several mutants were obtained by the process according to the invention from the industrial polyploid strain S47 which is deposited at C.N.C.M. under the n° I−2037. This strain was selected because it is the strain most currently used in France for bread-making with frozen doughs.

These mutants are the strains:

AT25=S47 fil400

AT26

AT28=S47 fil500

AT31.

The two strains AT25 and AT28 have been completely studied leading to the conclusion that there are a priori two different mutations The strain AT25 allows a direct use as an industrial baker's yeast strain for application to frozen doughs.

In a general fashion, the invention is not limited to the isolation of genes carrying the mutation in laboratory fil strains. It also encompasses the same process for isolating the gene carrying the mutation in industrial segregants or in industrial strains. In polyploid industrial strains, the mutation is probably dominant and consequently the strategy for isolation of the gene or genes concerned will have to be adapted. In this case, a genomic DNA library is constructed of the mutant fil in a centromeric vector such as the vector Ycp50, usually available, which contains the marker URA3. A laboratory yeast strain auxotrophic for uracil is transformed with this DNA library and, among the transformants which do not need uracil, are selected those which have acquired the fil phenotype, by the technique explained concerning the fil mutants described above.

It is remarkable that the different fil mutants obtained do not have mutations which affect the same metabolic pathway. This is indicated by the following table:

Strains

| | | | | AT25; AT26; |
|---|---|---|---|---|
| Properties | fil1 | fil2 | fil300 | AT28; AT31 |
| Reduction of | yes | yes | yes | yes |
| the loss of | | | | |
| resistance | | | | |
| to stress | | | | |
| induced by | | | | |
| fermentation | | | | |
| Deficiency | yes | yes | yes | no |
| of cAMP signal | | | | |
| induced by | | | | |
| glucose | | | | |
| Deficiency of | partially | no | n.d. | n.d. |
| accumulation of | yes | | | |
| cAMP induced by | | | | |
| acidification | | | | |
| Level of | yes | yes | yes | yes |
| trehalose | | | | |
| increased | | | | |
| Normal latency | yes | yes | yes | yes |
| phase for culture | | | | |
| on glucose | | | | |
| Normal | yes | yes | yes | yes |
| growth | | | | |
| rate on | | | | |
| glucose | | | | |
| Normal CO₂ | yes | yes | yes | yes |
| production | | | | |
| in the dough | | | | |
| Normal | yes | yes | yes | yes |
| harvest on | | | | |
| molasses | | | | |

n.d.: not determined

The increase in cAMP level induced by the glucose is affected in the mutants fil1, fil2 and fil300. This is not however the case in the mutants fil400 (AT25), fil500 (AT28), and in the mutants AT26 and AT31.

In the case of the mutants fil1, fil2 and fil300, the mutations affect the Ras-cAMP-PKA pathway. This is entirely normal for the fil1 mutation. This leads to the conclusion that the gene GPR1, whose function is not well known, has a function in this pathway.

In the case of the other mutants, the mutation does not seem to concern the Ras-cAMP-PKA pathway.

It is known that the Ras-cAMP-PKA pathway is not the only pathway implicated in the mechanisms of resistance to stress. The new process according to the invention is a tool which is particularly interesting for:

isolating new mutants resistant to stress

characterizing the gene or genes concerned in different metabolic pathways

constructing with the help of these genes new industrial strains which are particularly efficient.

The resistances to stress are often crossed. The same metabolic pathways are often found in a number of different eukaryotes. In particular, pathways equivalent to the Ras-cAMP-PKA pathway have been described in numerous eukaryotes. Consequently, the present invention is not limited to the obtaining of fresh or dry yeasts for bread-making, for brewing, for winemaking, for alcohol production and distillation, for the production of heterologous proteins, but to the obtaining of all new eukaryotic strains of interest for all industrial uses, such as the production of organic acids, amino-acids, enzymes, etc. . .

The present invention is also illustrated by the following examples. The list of figures concerning these examples is given hereafter. These figures are designated by the following nomenclature. The first number is the number of the example in which the figure is described, the second number is its order in the said example.

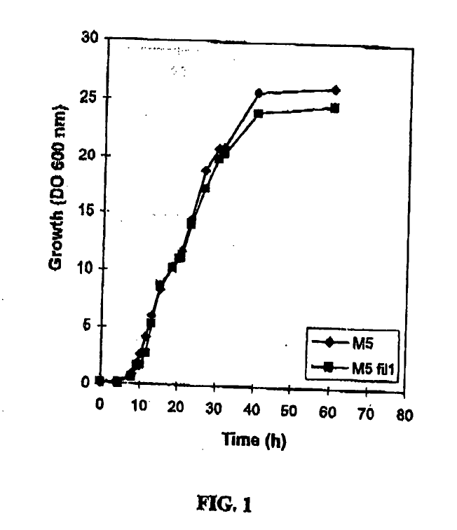

FIG. 1-1: Growth of the strains M5 and M5 fil1 =PVD1150 on YPD medium at 30° C. and under stirring (180 rpm) according to test T3.

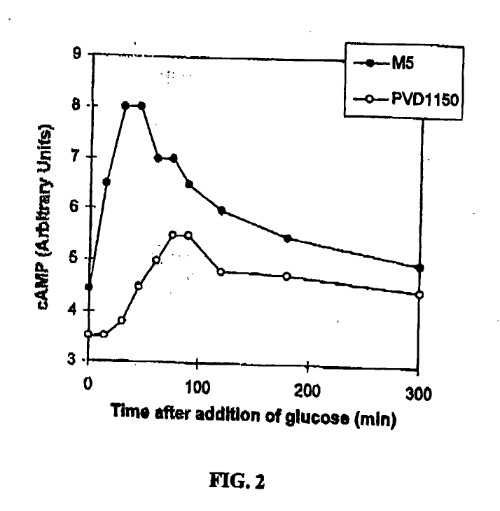

FIG. 1-2: cAMP response (Arbitrary Units) of the strains PVD1150 and M5 after addition of glucose (100 mM) to cells cultured on glycerol medium until obtaining of stationary phase.

FIG. 1-3: Monitoring of the degradation of trehalose (Arbitrary Units) in the strains PVD1050, PVD1150 and their respective controls M5 hxk2Δ and M5, after an induction by glucose (200 mM) on cells in stationary phase.

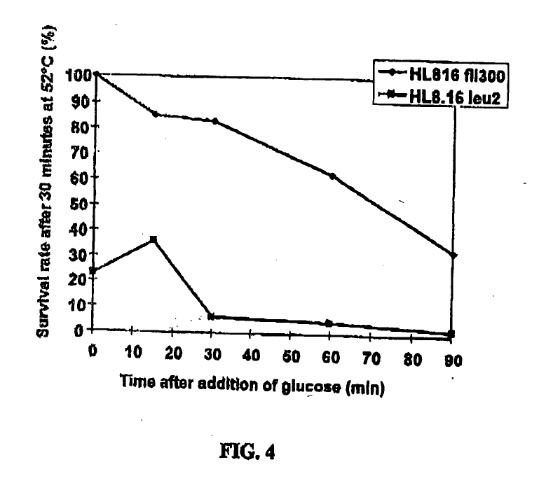

FIG. 1-4: Survival rate of the strains HL8.16 leu2 and HL816 fil300 after a thermic shock of 30 minutes at 52° C. according to test T1.

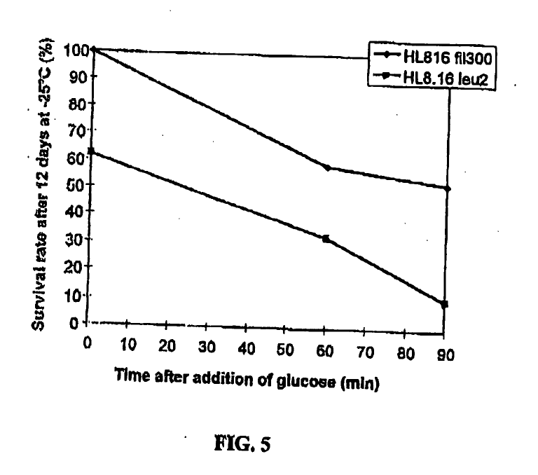

FIG. 1-5: Survival rate of the strains HL8.16 leu2 and HL816 fil300 after a freezing of 12 days at −20° C. according to test T2.

FIG. 1-6: Trehalose content (Arbitrary Units) in the strains HL8.16 leu2 and HL816 fil300. The strains are grown until obtaining of stationary phase, and then glucose (100 mM) is added at t=0.

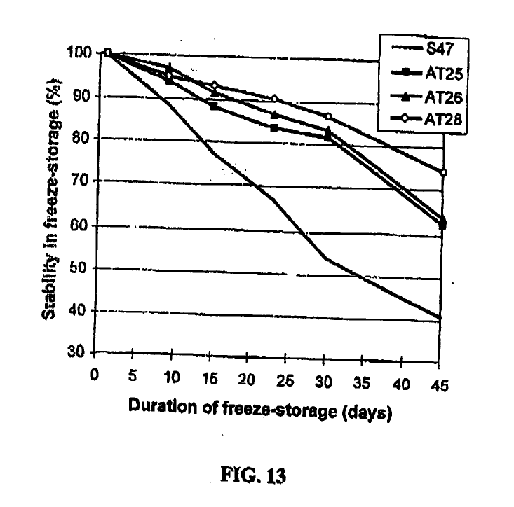

FIG. 1-7: cAMP response (Arbitrary Units) after induction by glucose (100 mM) in the strains HL8.16 leu2 and HL816 fil300 cultured until obtaining of stationary phase. The initial adding of 3 mM of glucose allows to avoid the cAMP response related to intracellular acidification.

FIG. 2-1: Monitoring of the degradation of trehalose (Arbitrary Units) after an induction by glucose at t=0 (on cells of strains S47, AT25, AT26, AT28, AT31 in stationary phase).

FIG. 2-2: Monitoring of the cAMP response (Arbitrary Units) after an induction (at t=0) by glucose on cells in exponential growth phase on maltose. a) control S47 and mutant AT25; b) control S47 and mutants AT26, AT28 and AT31.

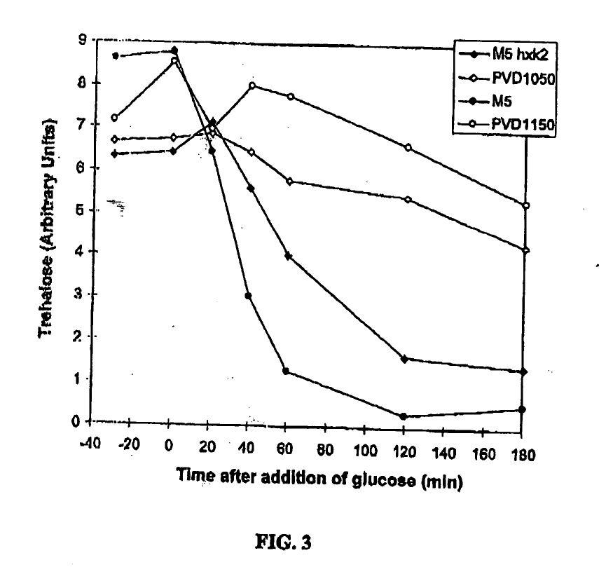

FIG. 3-1: Gap-filling strategy (filling of missing DNA) used for isolating the gene carrying the fil1 mutation in the strain PVD1150.

FIG. 3-2: Physical map of the vector pUC18-CYR1mut-URA3 [Sn].

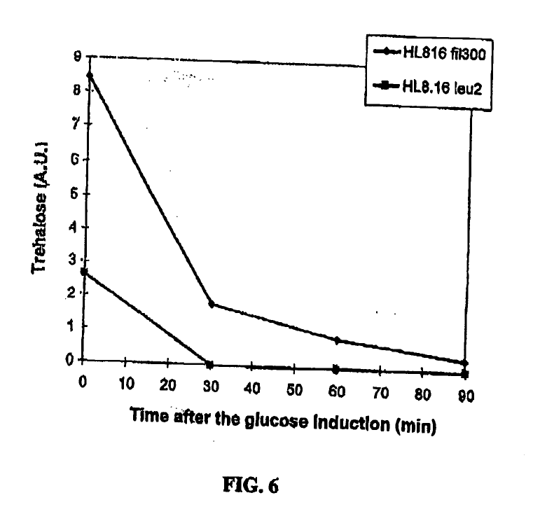

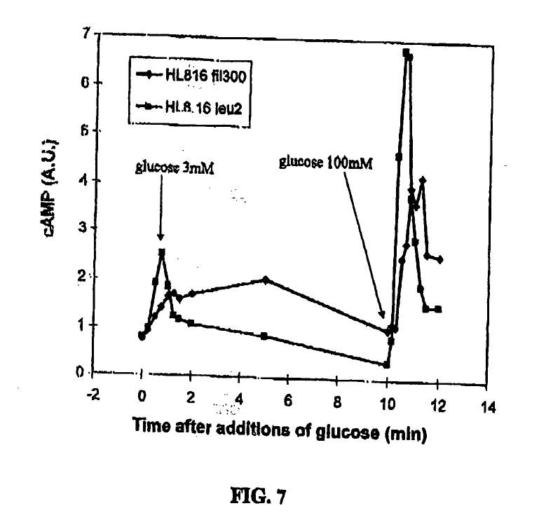

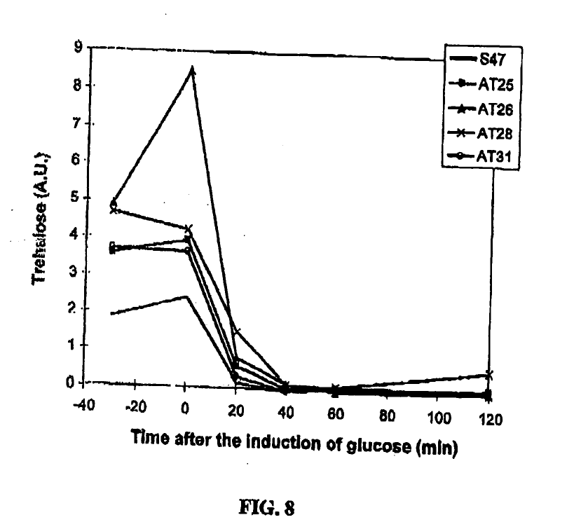

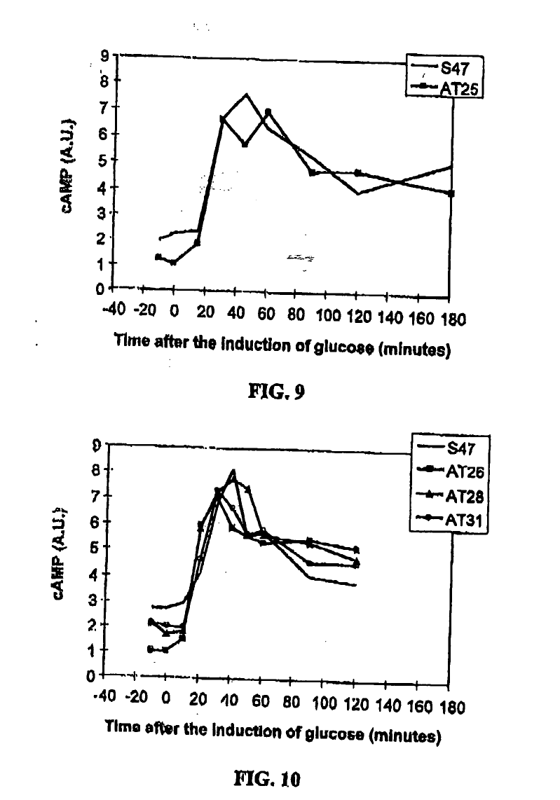

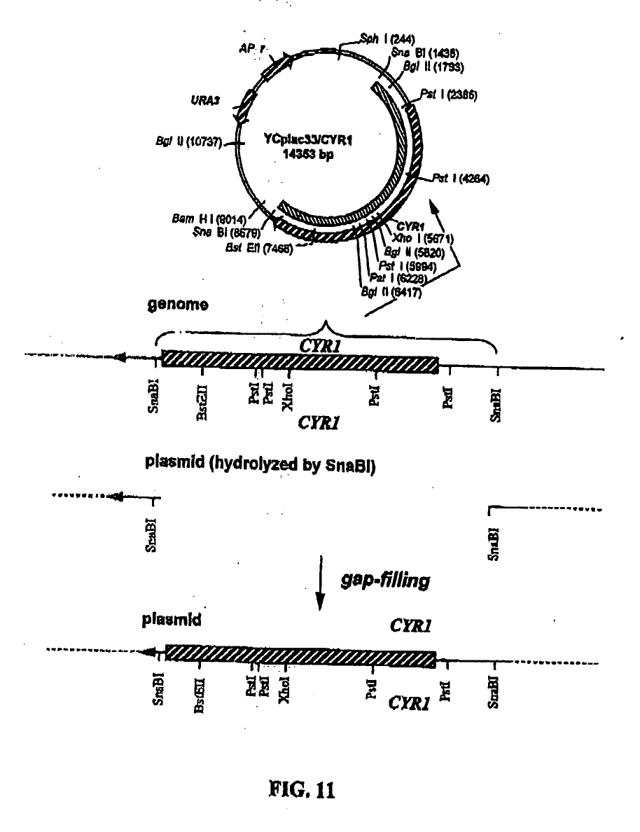

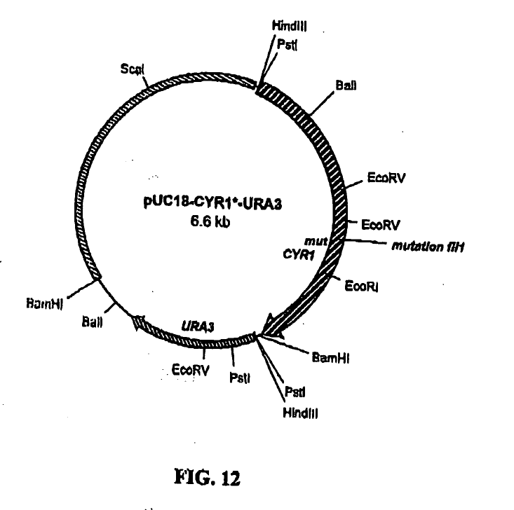

FIG. 6-1: Stability to freezing measured by the ratio between the gassing power after 1 day of conservation at −20° C. and n days of storage at −20° C.

### EXAMPLE 1

### Use of Heat Stress for the Isolation of fil Mutants

A. Obtaining of Strains having the fil1 Phenotype

a) Obtaining of the Strains PVD1050 and PVD1150

The starting strain is the haploid strain M5 hxk2Δ (strain comprising a deletion of the gene of hexokinase II) which is derived from the haploid strain M5, which is derived from the diploid strain M5 (Schaaff et coll. (1989), Curr. Genet. 15: 75-81). A mutagenesis using E.M.S. (Ethyl Methyl Sulfonate) has been carried out according to the technique disclosed in “Methods in Yeast Genetics, Cold Spring Harbor Laboratory Press” (Sherman et coll., 1986) in such a way as to obtain a survival rate of about 10%. After this treatment, the cells are washed and resuspended in the YPD medium. They are then cultivated until stationary phase is reached. A 0.25 ml sample of this culture has been taken and used for the inoculation of 25 ml of YPD medium. The inoculated medium has been incubated at 30° C. for 90 minutes, and then heated to 52° C. for 30 minutes. It has been then incubated at 30° C. for 24 hours, and then 0.5 ml of the culture were transferred into 25 ml of YPD medium. This medium has been incubated at 30° C. for 90 minutes. Fractions of 100 μl of this medium were then taken and placed at 56° C. for 30 minutes. Then, the cell suspension was spread on YPD-A medium. The stress resistance was then evaluated by tests on surviving cells forming colony on YPD-A medium.

In order to test the said resistance, the yeast cells of the strains as obtained were tested according to the test T1, using an incubation at 52° C. for 30 minutes. Under the conditions of this test and after verification of the conservation of the growth properties and of the fermentation properties, as well as of the stability of the mutation, one strain has been selected and called Mut1 or PVD1050. It has been shown that this strain comprises a stable mutation, which was called fil1 and which is monogenic and recessive. The haploid strain PVD1050 has a survival rate after thermic treatment which is surprisingly high, and this high survival rate is observed for stationary cells and also for cells growing in exponential phase, as shown by the results collected in table 1-A. It is surprising that, in the case of a Saccharomyces cerevisiae strain, 100% viability is conserved in exponential phase after a thermic treatment of 30 minutes at 52° C. It is also surprising to notice that this strain PVD1050 is not significantly penalized from the standpoint of growth and fermentation with respect to the strain M5 hxk2Δ, being recalled that the deletion of the gene HXK2 penalizes among others the growth.

In order to eliminate the HXK2 deletion from this strain, the haploid strain PVD1050 (=M5 hxk2Δ fil1) has been crossed with the haploid strain M5 and subsequently tetrads were dissected. In the thus obtained haploids, a strain presenting the fil1 mutation but having also a wild type HXK2 gene was selected. The thus isolated haploid strain was called PVD1150 or M5 fil1. That strain has the same thermoresistance phenotype as the strain PVD1050, i.e. a survival rate which is extremely high in the test T1 (table 1-A).

TABLE 1-A
EVALUATION OF THE SURVIVAL RATE OF
fil1 STRAINS AND OF THEIR CONTROLS
AFTER A THERMIC SHOCK OF 30 MINUTES AT 52° C.
Test T1 with a survival rate after 30′ at 52° C. (%)

| | In stationary | In active metabolism = glucose incubation of | | |
|---|---|---|---|---|
| | Phase | 30′ | 60′ | 90′ |
| M5 hxk2Δ | 70% | 40% | 30% | 15% |
| PVD1050 | 100% | 98% | 98% | 98% |
| M5 | 50% | 20% | 10% | 5% |
| PVD1150 | 99% | 96% | — | — |

b) Characterization of the strain PVD1150

The physiological and genetic characterization of the fil1 mutation was carried out on this new haploid strain M5 fil1, the haploid strain M5 being the control.

In a first step, the growth of the strain PVD1150 on glucose has been compared with that of the control M5 in order to study the possible influence of the fil1 mutation on the growth. The strains have been cultured on YPD medium according to the conditions of test T3. The results (FIG. 1-1) clearly show that the fil1 mutation very slightly affects the growth of the strain M5. These same strains are then cultivated on molasses agar according to the conditions of test T5. An identical growth yield has been obtained in the case of the two strains (table 1-B).

The harvested yeasts have been used in order to carry out the measurement of the fermentative ability under the conditions of test A20. This test shows that the loss in gassing power associated with the fil1 mutation does not exceed 20% (table 1-B).

TABLE 1-B

| | Test T5 | |
|---|---|---|
| | Harvest (expressed in dry matter of | Test A20 |
| | yeast) after 40 h [with respect | Fermentative ability |
| | to 8 g of molasses] | (CO₂ ml released in 2 hours) |
| M5 | 1.70 g | 66-75 ml |
| M5 fil1 | 1.74 g | 60-65 ml |

From this table, it appears that there is no penalization during growth and a low penalization or decrease of the fermentation as far as the mutant is concerned. It results that the mutant is a mutant which presents very likely the new fil phenotype, object of the invention.

In the case of the two strains presenting the fil1 mutation (PVD1050 and PVD1150) the levels of cAMP and of trehalose were examined with respect to their respective controls (M5 hxk2Δ and M5). The cAMP level has been measured on the strains PVD1150 and M5, according the method disclosed by Thévelein et coll., 1987, J. Gen. Microbiol., 133, pp.2197-2205. The cAMP level has thus been determined after induction of its synthesis by addition of glucose (100 mM) on cells which have reached the stationary phase after growth on glycerol. An attenuated cAMP signal has been shown in the mutant PVD1150 (FIG. 1-2). Trehalose has also been determined on the strains PVD1150, PVD1050, M5 and M5 hxk2Δ, according to the conditions described by Neves et coll., 1991, FEBS Lett., 283, pp.19-22. In the case of strains presenting the fil1 mutation, the mobilization of the trehalose (after induction by glucose on cells in stationary phase) is far less rapid than in the control strains (FIG. 1-3).

From the results of the determination of trehalose and of cAMP, it seems obvious that the fil1 mutation concerns the Ras-cAMP pathway. However, and in contradiction with respect to the mutants of the Ras-cAMP pathway previously isolated and which show growth delays and fermentation delays , the fil1 mutant is only slightly affected during growth and fermentation.

In order to determine the actual influence of the increase trehalose content on the stress resistance of mutants fil1, the gene TPS1 (which codes for the synthesis of trehalose phosphate starting from glucose) has been deleted in the strains fil1 and in the starting control strains. Despite the fact that these strains fil1 tps1Δ no longer synthesize trehalose, they retain a much better resistance against thermic stress than the control strains tps1Δ: 30% survival after 30 minutes at 52° C., the survival being 0.1% as far as the controls are concerned (conditions of test T1). This confirms the results published by Van Dijck et coll. (1995, Appl. Environ. Microbiol,. 61, pp.109-115) which demonstrated the fact that trehalose is not the sole responsible for the improvement of stress resistance.

B. Isolation of strains fil300

a/ Isolation of the Strain FD51=HL816 fil300

A mutagenesis has been carried out on strain HL8.16 leu2 (aneuploid strain, segregant of an industrial strain made auxotrophic for leucine; LESAFFRE's collection): the cells have been cultured at 30° C. in a YPD medium until the stationary phase was obtained. Then the cells have been incubated at 30° C. for 1 hour, in the presence of E.M.S. (Ethyl Methyl Sulfonate) according to the technique disclosed in “Methods in Yeast Genetics, Cold Spring Harbor Laboratory Press” (Sherman et al., 1986) in such a way that a survival rate of about 10% is obtained. After that treatment, the cells have been washed, resuspended in YPD medium and cultured at 30° C. until the stationary phase was obtained. Glucose has then been added in the culture in stationary phase in order to obtain a final concentration in glucose equal to 100 mM. The inoculated medium has been incubated at 30° C. for 30 minutes, then at 56° C. for 30 minutes, and finally at 65° C. for 30 minutes. The surviving cells have been isolated by spreading on YPD-A medium. Then they have been subjected individually to a test of resistance to heat, according to the conditions of test T1 with an incubation of 30 minutes at 52° C. At the end of this first selection, the six strains which presented the highest survival rates have been retained and subjected to a second mutagenic treatment, with ultraviolet rays (U.V.), according to the technique disclosed by Spencer J. F. T. and Spencer D. M., Chapter “Yeast genetics” extract of “Yeast a practical approach”, 1988, Campbell and Duffus Eds. For this treatment, the cells of each of the six strains have been cultivated in a YPD medium until obtaining of the stationary phase. They were then washed with water, diluted and spread on YPD-A medium. U.V. light at a dose of 30 mJ (wavelength equal to 260 nm) has been applied on the open dishes. The 248 colonies which survived the mutagenic treatment were transferred to a YPD-A medium and then incubated during 2 hours at 60° C. At the end of this thermic treatment, only 2 colonies survived. A test of resistance to heat in liquid medium has then been carried out on these two colonies, according to the conditions of test T1 with an incubation of 30 minutes at 52° C. after an incubation in the presence of glucose for 1 hour at 30° C. The best of the 2 strains was subjected to a last mutagenic treatment with U.V. light (ultraviolet rays), according to the conditions previously disclosed however with a dose of 10 mJ of U.V. light (260 nm). The colonies which survived the mutagenic treatment were transferred to a YPD-A medium and incubated during 6 hours at 60° C. At the end of this thermic treatment, 171 colonies (of close to 1500) survived. A heat resistance test carried out on these 171 strains according to the conditions of test T1 permitted the selection of strain FD51, also called HL816 fil300. It has of course been checked that the thus obtained mutation is stable and that it corresponds to all of the conditions of fil phenotype.

b/ Characterization of the Strain HL816 fil300

A significant improvement of the heat resistance of the strain has been confirmed by test T1, either in stationary phase or in active fermentation phase (FIG. 1-4). Furthermore, mutation fil300 induced a resistance against other stresses: strain HL816 fil300 presents, under conditions of test T2, a survival rate of more than 50% after freezing during 12 days at −20° C. of prefermented cells during 90 minutes at 30° C. while the survival rate of the control is in the same conditions lower than 11% (FIG. 1-5). The strain HL816 fil300 presents consequently a much better resistance against freezing than the control strain, whether the cells are in stationary phase or in active fermentation phase.

In order to better characterize the phenotype linked to the fil300 mutation, the levels of trehalose and cAMP have been determined on cells in stationary phase which were subjected to an induction by glucose. The control strain is the starting strain HL8.16 leu2. During stationary phase (i.e. 0 minute of fermentation in FIG. 1-6), the trehalose level is 3 to 4 times higher in mutant fil300 than in control HL8.16 leu2. During exponential phase, the degradation rate of trehalose in the mutant HL8.16 fil300 strain is reduced, with respect to that of the control (FIG. 1-6). In mutant HL816 fil300 a cAMP response reduced by close to 50% after an induction of the synthesis by glucose on cells in stationary phase has also been observed (FIG. 1.7).

The performances of growth and of fermentative ability of strains HL8.16 leu2 (control) and HL816 fil300 were determined according to the tests T5 and A20. The results are presented in table 1-C. The strain fil300 retains in these tests 80% of the characteristic features measured for the starting strain.

TABLE 1-C

| | T5 | A20 |
|---|---|---|
| | Harvest (in dry matter of yeast) | Fermentative ability |
| | 20 h [for 8 g of molasses] | (ml CO₂ released in 2 hours) |
| HL8.16 leu2 | 1.5 g | 85 |
| HL816 fil300 | 1.2 g | 68 |

b/ Isolation of the Strain HL822 fil300

The decreases of biomass growth yield and of fermentative ability of mutant HL816 fil300 which are at the maximum of what is tolerated within the frame of fil phenotype are detrimental to its utilization. This is the reason why crossings have been carried out between the said mutant and some segregants in order to isolate in the descendance of the diploids obtained, thermoresistant segregants having improved performances. The strain HL822 fil300, also called FDH16-22, has thus been isolated. This strain, which is a segregant issued from a crossing between the strain HL816 fil300 and the segregant HL816 (LESAFFRE's collection), has a growth yield on molasse and a gassing power identical to those of the control strain HL8.16 leu2 (table 1-D). It presents furthermore a level of thermoresistance identical to that of the strain HL816 fil300.

TABLE 1-D
GROWTH YIELD ON MOLASSES AND FERMENTATIVE
ABILITY (DETERMINED IN TEST A20) OF THE
STRAINS HL8.16 LEU2 AND HL822 FIL300

| | T5 | A20 |
|---|---|---|
| | Harvest (in dry matter of yeast) | Fermentative ability |
| | after 20 h [for 8 g of molasses] | (ml CO₂ released in 2 hours) |
| HL8.16 leu2 | 1.5 g | 85 |
| HL822 fil300 | 1.5 g | 85 |

With respect to each of the two fil300 strains (HL816 fil300 and HL822 fil300), the stability under the form of frozen doughs has been compared with that of the control strain HL8.12 leu2 by monitoring the gassing power as a function of freezing duration under the conditions of test C2.

A significant improvement of the stability during freezing has been observed for the fil300 strains with respect to the control HL8.16 leu2 (table 1-E). Thus, the fil300 strains retain at least 60% of their fermentative ability (=gassing power) after 1 month of conservation at the temperature of −20° C. while the control only conserves 40% of its gassing power under the same conditions (table 1-E).

TABLE 1-E
STABILITY DURING FREEZING, DETERMINED BY THE
RATION BETWEEN THE FERMENTATIVE ABILITY AT DAY
N OF MEASUREMENT AND THE FERMENTATIVE ABILITY
AFTER THE FIRST DAY OF FREEZING (TEST C2)

| | Test C2 | | |
|---|---|---|---|
| | Stability during freezing (%) | | |
| | 1 day | 28 days | 42 days |
| HL8.16 leu2 | 100% | 41% | 39% |
| HL816 fil300 | 100% | 60% | 53% |
| HL822 fil300 | 100% | 62% | 57% |

### EXAMPLE 2

### Use of the Cycles of Freezing/Thawing for Isolation of fil Mutants Starting from an Industrial Strain

The starting or original strain is the strain S47 deposited at the C.N.C.M., 25 rue de Docteur Roux, 75724 Paris, France, under the number I−2037. This strain is an aneuploid baker's yeast strain used industrially, it is probably the best strain at present used in France for making raw frozen doughs. It has been grown in a liquid YPD medium at 30° C. until obtaining of the stationary phase. A mutagenic treatment with U.V. was carried out according to the technique described by Spencer J. F. T. and Spencer D. M., Chapter “Yeast genetics” extract of “Yeast a practical approach” (1988), Campbell and Duffus Eds., in order to obtain a survival rate of about 10%. The cells have thus been treated with a dose of U.V. (260 nm) of 5 mJ. They have then been grown at 30° C. on a medium consisting of agar YE-molasses (agar 20 g/l, extract of yeast 5 g/l, molasses 5 g/l, (NH₄)₂HPO₄ 0.5 g/l, pH 5.0-5.5), and were then harvested after 2 to 3 days by washing of the dishes with water. Mini-pieces of dough of 0.5 g were prepared with the harvested yeasts, with the following composition: flour 56.5%, NaCl 1.0%, water 42.5%, yeasts 4.10⁸ cells/gram of dough. These mini-pieces of dough were incubated at 30° C. for 30 minutes. Then they were subjected to 200 successive cycles of freezing at −30° C. and thawing at room (ambient) temperature, in order to obtain only a few thousand surviving cells (survival rate lower than 0.01%), which were isolated from the pieces of doughs.

The freeze resistance has then been tested individually on the surviving cells, by determination of survival rate of the cells after one freezing and by determination of the loss of fermentative ability (=gassing power) after freezing. In parallel, the growth yield on molasses and the fermentative ability (=gassing power) of the best strains have been measured.

The survival rate after freezing is measured according to the conditions of test T2 with a fermenting phase of 30 minutes at 30° C., followed by a freezing at −30° C. for 24 hours, and then by a thawing at room temperature.

The growth of the strains has been measured by the yield obtained after a culture for 20 hours on agar molasses medium according to test T5; the harvested yeasts are used to determine the fermentative ability at 30° C. and over 2 hours, according to the conditions of test A1.

From the strains which survived the successive treatments of freezing/thawing in mini-pieces of dough, 7 strains named AT25, AT26, AT27, AT28, AT29, AT30 and AT31 were pre-selected. The results of the survival rates after freezing (test T2) are collected in table 2-A, the molasses growth yields (T5) and the gassing power (A1) are collected in table 2-B. Even if all of these mutants present a survival rate (table 2-A) much better than that of the control, it is necessary to take into account the growth yield and gassing power of each mutant for the selection of strains which correspond to criteria of fil phenotype (table 2-B). This is the reason why only the strains AT25, AT26, AT28 and AT31 are selected at the end of these tests. The measurements of the maximal growth rate were carried out according to test T4 on these different mutants and the control strain S47, the results being indicated in table 2-C.

TABLE 2-A

Test T2
Survival rate (%) after:

| | | Freezing (−25° C. |
|---|---|---|
| | Freezing without previous | during 24 hours) with |
| | fermentation −25° C. | previous fermentation |
| | during 24 hours | of 30 minutes at 30° C. |
| S47 | 36 | 17 |
| AT25 | 51 | 47 |
| AT26 | 59 | 42 |
| AT27 | 45 | 40 |
| AT28 | 47 | 39 |
| AT29 | 72 | 32 |
| AT30 | 47 | 28 |
| AT31 | 38 | 27 |

TABLE 2-B
GROWTH YIELD (EXPRESSED BY THE RATIO BETWEEN
THE AMOUNT OF YEAST PRODUCED IN 20 HOURS EXPRESSED
AT 30% OF DRY MATTER WITH RESPECT TO THE AMOUNT
OF MOLASSES USED CONSIDERED AS BEING 50% OF SUGAR)
AND FERMENTATIVE ABILITY (EXPRESSED BY THE AMOUNT
OF CO₂ RELEASED IN 2 HOURS AT 30° C.)
OF THE MUTANTS OF THE STRAIN S47 AND OF THE STARTING
STRAIN S47

| | | Test A1 |
|---|---|---|
| | Test T5 | Fermentative |
| | Growth | ability |
| | Yield % | (ml CO₂) |
| S47 | 67% | 138 |
| AT25 | 64% | 116 |
| AT26 | 62% | 116 |
| AT27 | 47% | 113 |
| AT28 | 62% | 120 |
| AT29 | 51% | 64 |
| AT30 | 62% | 84 |
| AT31 | 62% | 133 |

TABLE 2-C
MAXIMAL GROWTH RATE OF STRAIN S47
AND OF THE ISOLATED MUTANTS DERIVED
FROM STRAIN S47, ON DIFFERENT GROWTH
MEDIA ACCORDING TO TEST T4

| | MAXIMAL GROWTH RATE | | |
|---|---|---|---|
| | or μmax (h⁻¹) | | |
| STRAIN | glucose 100 mM | glucose 10 mM | molasse 0.5% |
| S47 | 0.69 | 0.60 | 0.57 |
| AT25 | 0.60 | 0.61 | 0.54 |
| AT26 | 0.67 | 0.56 | 0.56 |
| AT27 | 0.37 | 0.23 | 0.26 |
| AT28 | 0.61 | 0.62 | 0.60 |
| AT29 | 0.59 | 0.48 | 0.52 |
| AT30 | 0.62 | 0.55 | 0.56 |
| AT31 | 0.63 | 0.60 | 0.57 |

In order to check whether the mutations carried by the strains AT25, AT26, AT28 and AT31 also affect the Ras-cAMP pathway as in the case of the fil strains described in example 1, the levels of cAMP and of trehalose in these different strains have been measured. The measurements of trehalose have been carried out on cells cultured until the stationary phase and then subjected to an induction of its mobilization or degradation by addition of glucose (100 mM). The initial level of trehalose, which corresponds to the level of trehalose in stationary phase, is higher for all the mutants than for the control (t=0, FIG. 2-1). In the presence of glucose, the degradation of trehalose is rather quick for each of the said strains (mutants and control) as after 20 minutes and more, a low and almost identical level of trehalose is obtained for all of these strains. However, the stress imposed within the frame of test T2 is realized after an incubation of 30 minutes in the presence of glucose. This confirms, here again, that trehalose does not allow an explanation of the better performances of these fil mutants. As far as cAMP is concerned, the measurements were carried out on cells which were in exponential growth phase on maltose which have been subjected to an induction by glucose (100 mM). In these experiments, there is no significant reduction of the cAMP signal in the mutants with respect to the control strains S47 (FIGS. 2-2, a & b). Consequently, it seems that the cryoresistant mutants AT25, AT26, AT28 and AT31, which had been isolated following successive cycles of freezing/thawing and which have the fil phenotype, carry mutations which affect other targets than the Ras-cAMP pathway.

### EXAMPLE 3

### Identification of the fil1 Mutation and Reconstruction of Strains Carrying the fil1 Mutation

Crossings between the strain PVD1150 and other strains have shown that the fil1 mutation is located close to a centromere. The strain PVD1150 has consequently been recrossed with different haploid strains carrying genetic markers located close to the centromeres of each chromosome and analysis has been carried out on the tetrads originating from the sporulation of the thus obtained diploids.

It has been determined that the fil1 mutation is located close to the centromere of chromosome X. The strain PVD1150 has consequently been complemented with each of the genes located in that region, according to the following general strategy:

transformation of the strain PVD1150 with centromeric plasmids carrying each one of the genes located at less than 25 kb from the centromere of chromosome X,

search for the transformant clones having lost the phenotype of thermoresistance (=heat-resistance),

isolation of the gene carrying the mutation from the mutant PVD1150 by way of the technique of “gap-filling” hereafter defined and illustrated by FIG. 3-1; retransformation of the strain PVD1150 with the mutated gene thus isolated in order to verify that it is not a suppressor-gene,

sequencing of the mutated gene originating from PVD1150 and of its wild type allele originating from M5 in order to identify and to locate the mutation,

reintroduction by homologous recombination of the mutated gene in at least one other thermosensitive (=heat-sensitive) lab strain and verification of the obtaining of fil phenotype showing the monogenicity of the mutation and its non dependency upon the genetic context of the mutated original strain PVD1150.

This strategy of complementation has been carried out according to the following manner: all these constructions have been made according to the usual techniques and especially according to the book “MOLECULAR CLONING”, J. Sambrook, E. F. Fritsch, T. Maniatis, Cold Spring Harbor Laboratory Press, 1989.

1/ Transformation of the Strain PVD1150 by Centromeric Plasmids Carrying each one of the Genes Located at Less than 25 kb of the Centromere of Chromosome X

Each gene located at less than 25 kb from the centromere has been amplified by PCR, then cloned in the vector YCplac33 (Gietz R. D. and Sugino A. (1988), Gene, 74, pp.527-534) which carries the gene URA3. Each of the thus obtained vectors is used in order to transform the strain PVD1150 (auxotrophic with respect to uracil). The presence of the plasmids is then checked on the thus obtained transformants.

2/ Search for the Transformants having Lost the Phenotype of Thermoresistance

The different transformants carrying each, on a monocopy vector, one of the genes located at least at 25 kb from the centromere of chromosome X, have been tested for their loss of thermoresistance. Each strain PVD1150 carrying one of the genes located around the centromere of chromosome X is streaked on YPD-A medium preheated at 57° C., which is incubated for 90 minutes at 57° C. In parallel, the strain PVD1150 which contains a monocopy plasmid without insert, here the vector YCplac33, is used as non complemented control. After this thermic treatment, the strains are incubated at 30° C. until development of the growth of the control (appearance of a cellular layer). The strains carrying a gene which complements the fil1 mutation are those which have not yet developed.

The results of these tests allowed to demonstrate that the fil1 mutation is located in the gene CYR1 (also called CDC35), as only that gene was capable of complementing the fil phenotype in strain PVD1150. The gene CYR1/CDC35 codes for adenylate cyclase, enzyme of the Ras-cAMP pathway which permits the synthesis of cAMP starting from ATP. This result is coherent with the fact that the PVD1150 strain presents a reduced level of cAMP (example 1).

In order to check that the loss of thermoresistance is actually linked to the presence of the wild type gene CYR1/CDC35 introduced by the plasmid, the strain PVD1150 containing the vector YCplac33-CYR1 has been cultured on YPD medium, a medium which is non selective and which permits rapid loss of the plasmid. Colonies have been isolated from that culture on a YPD-A medium, and then replicated on selective medium SD-URA (medium deprived of uracil) in order to search for clones which do not grow and which consequently have lost the plasmid. A thermoresistance test identical to the previous one confirms that all these clones having lost the plasmid had again become thermoresistant.

3/ Isolation of the Gene Carrying the Mutation from the Mutant PVD1150

The deletion of the gene is known as having as consequence the lethality of the strains. It was necessary to isolate the mutated gene.

The mutated gene of the strain PVD1150 has been cloned by the technique of “gap-filling” [Iwasaki, T. and coll. (1991) Gene, 109, pp.81-87] or “allele rescue” [Orr-Weaver, T. L. et coll. (1983) “Methods Enzymol.”, 101, pp.228-245].

This method has been used with the strain PVD1150 by transformation with the plasmid YCplac33-CYR1 digested by the enzyme SnaBI (FIG. 3-1). The transformants which grow on minimum medium without uracil (medium SD-URA) have then been selected. These transformants can only grow when there has been a recircularization by the vector, especially coming from an event of double recombination having integrated the missing part of the gene due to the mutated gene CYR1 in the PVD1150 strain (FIG. 3-1). In parallel, the strain M5 has been transformed with the vector YCplac33-CYR1 digested by SnaBI, then the transformants have been selected on minimum medium in order to obtain a vector carrying the non mutated gene CYR1 originated from the control strain M5.

The strain PVD1150 has then been retransformed:

with the vector YCplac33-CYR1^{mut} which carries the mutated gene originating from vector YCplac33-CYR1 digested by SnaBI and filled by gap-filling in the strain PVD1150,

with the vector YCplac33-CYR1/S which carries the wild type gene originating from vector YCplac33-CYR1 digested by SnaBI and filled by gap-filling in the strain M5.

The level of thermoresistance of these two strains has then been compared with the help of the following test: streaks of cells YPD-A medium preheated at 57° C. and incubation at 57° C. for 90 minutes. As expected, the mutated gene does not complement the mutation; on the other hand, the wild type CYR1 gene makes the strain PVD1150 thermosensitive.

4/ Sequencing of the Mutated Gene (Originating from PVD1150) and of its Wild Type Allele (Originating from M5) in Order to Identify and Locate the Mutation

The mutated gene CYR1^{mut} originating from PVD1150 and the wild type CYR1 gene originating from M5 were sequenced in parallel by the technique of direct sequencing on PCR products obtained by amplification of fragments covering the whole gene, due to which it became possible to locate the fil1 mutation. It consists of the change of one base G into one base A in position n° 5044 of the coding part of gene CYR1 (gene referenced under the name YJL005w in the case of Saccharomyces cerevisiae).

This also corresponds to a change in position 429888 of chromosome X, according to the classification of MIPS (Munich Information Center for Protein Sequence). This change is represented in the following table.

| Control strain | fill strain |
|---|---|
| glu | lys |
| 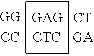 | 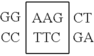 |

The mutation has been verified both by sequencing of a mixture of three independent PCR products and by sequencing of the complementary DNA strand. This change leads to the change from a glutamic acid into lysine in position 1682 of the protein. It occurs in the region coding for the catalytic site of the enzyme, and close to the region which is supposed to be involved in the activation of adenylate cyclase by the Ras proteins:

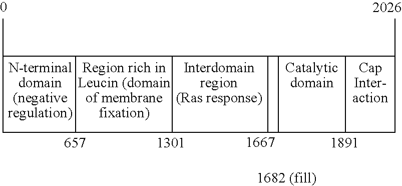

5/ Reintroduction (by Homologous Recombination) of the Mutated Gene in the Thermosensitive Lab Strains and Characterization of the Associated Phenotype

a/ Construction of the Plasmid pUC18-CYR1^{mut} -URA3 [Sn]

The fragment PstI-BamHI of the vector Ycplac33-CYR1^{mut}, fragment which contains the 3′ part of the gene CYR1^{mut} (including the fil1 mutation) has been sub-cloned in the vector pUC18. Then the auxotrophy marker URA3, originating from vector pJJ242 (Jones and Prakash (1990) Yeast, 6, pp.363-366) has been inserted in this new vector, downstream from the coding part of gene CYR1^{mut} in the 3′ non coding region, at the level of a unique restriction site (SnaBI). The obtained vector, called pUC18-CYR1^{mut} URA3 is represented in FIG. 3-3.

b/ Transformation of Haploid Strains of Yeasts M5 and SP1

The vector pUC18-CYR1^{mut}-URA3 has been hydrolyzed by the appropriate enzyme (here BalI), which allowed to obtain a linear fragment of 3.2 kb containing the fil1 mutation, the URA3 marker as well as the sequences located at the extremities permitting homologous recombination. This fragment was used for the transformation of lab strains: the strain M5 (ura3, trp1, leu2) which is a true haploid (n, i.e. 16 chromosoms) originating from the diploid strain M5 (2n) described by Schaaf and coll. (1989) Curr. Genet., 15, pp.78-81, and the strain SP1 (ura3, trp1, leu2, ade8) described by Toda and coll. (1985) Cell, 40, pp.27-36.

The presence of the mutation has then been searched for on some of the thus obtained transformants by sequencing of the zone carrying in principle the mutation. It became thus possible to isolate the strains M5 fil1 and SP1 fil1.

c/ Search for the Phenotype of Thermoresistance

The thermoresistance of the strains M5 fil1 and SP1 fil1 has been compared with that of their respective controls (M5 and SP1) according to the conditions of test T1 with an incubation in the presence of glucose for 30 minutes at 30° C. and a heat treatment of 20 minutes at 52° C.

The results concerning the reconstructed strains M5 fil1 and SP1 fil1, which are collected in table 3-A show that these strains actually present a phenotype of thermoresistance, which is not observed in the control strains.

TABLE 3-A
RESIDUAL VIABILITY AFTER THERMIC
SHOCK AT 52° C. OF THE RECONSTRUCTED
fill STRAINS AND OF THEIR CONTROLS

Residual viability after
treatment at 52° C. (%)

| Original strain | Wild type strain | fill strain |
|---|---|---|
| M5 | 9% | 96% |
| SP1 | 1% | 16% |

d/ Remarks

The fil1 mutation is a punctual mutation on the CDC35/CYR1 gene. A deletion of this gene is lethal, the cyr1 mutants previously described led to very low growth rates.

In the mutated fil1 strains, the mutated gene CDC35/CYR1 is overexpressed, to compensate the effect of the mutation, considering that the level of AMP cyclic remains low. The response to the fil1 mutation seems to correspond to a complex metabolite equilibrium, which may depend on the strain genetic background.

Furthermore, it must be noted that the fil1 mutation seems to delay in an important manner the germination of the spores containing this mutation. This deficiency concerning meiose cannot be found in growth or fermentation active phase. It must only by taken into consideration at the level of the constructs of fil1 strains by classical genetics.

### EXAMPLE 4

### Characterization of the fil2 Mutation

The strain KL1(=W303 fil2) has been isolated from the strain W303-1A (Thomas and Rothstein (1989) Cell, 56, pp.619-630) according to a research protocol of fil strains similar to those developed in example 1. The fil2 mutation is monogenic and recessive. In a first step, a thermoresistance test permitted to confirm the improvement obtained by fil2 mutation. Then a cross-resistance with respect to other stresses has been shown. It was checked that the mutated strain fil2 was actually a fil mutation as that mutated strain was not significantly affected when its growth and its fermentative ability were compared with those of the original strain W303-1A. Due to the fact that it is a lab strain, the gene carrying the mutation has been searched for.

a/ Resistances to Stresses

In order to measure the resistance to thermic stresses (heat and freezing), the strains KL1 and W303-1A were cultured on YPD medium at 30° C. until obtaining of stationary phase. After centrifugation, the cells were resuspended in YP medium and incubated at 30° C. After 30 minutes, glucose has been added until obtaining a final concentration of 100 mM in the medium. In the case of the heat stress, the incubation at 30° C. has been pursued for 30 minutes, and then the cells were incubated at 52° C. for 30 minutes. In the case of freezing, the incubation at 30° C. has been pursued for 90 minutes, and then the cells were resuspended in ice cold YP medium and frozen at −30° C. for 2 days and then thawed at 30° C. Four successive cycles of freezing/thawing have been carried out.

These two tests made possible to show that there is a correlation between the thermoresistance and the resistance against freezing in the case of the fil2 mutation. The strain KL1 has a survival rate of 80% after heat treatment while the survival rate of the control strain W303-1A is of 20%; furthermore, after freezing in connection with the severe test which has been carried out, the survival rate of the KL1 mutant is almost 4 times higher than that of the control: about 30% for KL1 with respect to about 8% for W303-1A.

b/ Determination of the Mutated Gene

This determination was carried out using a strategy which is identical to that with which it had been possible to identify the fil1 mutation (cf. example 3). It has thus been determined that the fil2 mutation was located close to the centromere of chromosome IV. The strain KL1 was consequently complemented with each of the genes located in that region, according to the general strategy disclosed in example 3

transformation of the strain KL1 (auxotrophic for uracil) by centromeric plasmids carrying each one of the genes located at less than 60 kb from the centromere of chromosome X

search for the transformant clones having lost the phenotype of thermoresistance

isolation of the gene carrying the mutation starting from mutant KL1; retransformation of the strain KL1 with the thus isolated gene in order to verify that it is not a suppressor gene.

The results of the complementation tests have permitted to show that the fil2 mutation was located in the GPR1 gene. This gene codes for a potential receptor coupled to a G protein and it is supposed to initiate the signal pathway associated with G protein encoded by the gene GPA2 (Xue, Y and coll. (1998) EMBO Journal 17: 1996-2007).

In order to verify that the phenotype of thermoresistance was actually associated with a mutation on the gene GPR1, the level of thermoresistance of the strain KL1 has been compared with that of the strain W303-1A in which the gene GPR1 has been deleted (non lethal deletion), as well as with that of the thermosensitive control W303-1A. The results clearly show that the deletion of the GPR1 gene permits obtaining of a high level of thermoresistance. Furthermore, the diploid obtained by crossing between KL1 and W303-1A gpr1Δ presents a level of thermoresistance as high as that of the strains KL1 and W303-1A gpr1Δ, which confirms that the fil2 mutation actually concerns the GPR1 gene.

The GPR1 gene mutated in order to provide the fil2 phenotype can be isolated according to technique described in example 4 and it permits the transformation of industrial strains in such a way as to obtain industrial strains having fil2 phenotype.

In the fil2 allele of GPR1 gene, the following mutation point has been found: the base 948 starting from the start codon, that is to say the ATG, is changed, a Thymine (T) becoming an Adenine (A). The codon in the wild type strain is TAT which codes for a Tyrosine in position 316 in the protein, it is changed in TAA which codes a STOP. The fil2 allele seems to code for a truncated version of the Gpr1 protein which contains 962 aminoacids. Aminoacid 316 before which the synthesis of the truncated fil2 protein stops, is located in the 3^{rd} intracellular loop of the receptor protein Gpr1. It is that loop which is known for its interaction in the receptor proteins of mammals with G protein, it is probably that loop which interacts in the yeast with the Gpa2 protein.

c/ Reintroduction of the Mutation in a Diploid Strain

A true diploid strain, not auxotrophic, relatively close to the industrial strains, has been transformed so that its two genes GPR1 are replaced by the mutated gene, corresponding to the fil2 allele. The starting diploid strain and the fil2 diploid strains have been grown on molasses in fed-batch, in pilot fermentors. A drying test has been performed with the obtained strains as well as a frozen dough stability test of C1 type. The result after drying is a slight decrease of the loss of gassing power with the fil2 diploid strain compared to the starting strain. A clear improvement by about 1.5 times of the frozen dough stability is shown after 15 days at −20° C.

### EXAMPLE 5

### Performances of fil Strains During Drying

The fil phenotype is interesting for obtaining more active dry yeasts, due to the fact that it permits to subject to drying more active biomasses, without dramatical loss of fermentative ability during drying.

The two reconstructed fil1 strains, obtained as disclosed in example 3 and the strain S47 fil500 (=AT28) obtained as described in example 2 and the corresponding starting or original strains have been cultured according to the classical processes in fed batch in pilot fermentor of some liters as described in U.S. Pat. Nos. 4,318,929, 4,318,930 and 4,396,632 in such a way that an active biomass having more than 7% of nitrogen per dry matter is obtained. This biomass is then dried using a process of fluidization type as disclosed in the above-identified US patents.

a/ Drying of the 2 Reconstructed fil1 Strains and the 2 Original Strains M5 and SP1

The fermentative ability of the biomass obtained with each of the 4 strains before drying, i.e. in the form of fresh yeast having about 30% of dry matter has been measured according to test A20. The fermentative ability of the dry yeasts having about 95% of dry matter has been measured according to the test A′20. Due to the fact that, within the frame of such a pilot test, the raw gassing power values do not have any significance, the loss during drying obtained with each original strain has been affected with a coefficient 100 and the loss during drying of the reconstructed fil1 strains obtained according to example 3 expressed in percentage of loss during drying of the starting strain. These results are given in table 5A.

TABLE 5-A
EVALUATION OF THE LOSS OF GASSING
POWER AFTER DRYING OF THE fill STRAINS
RECONSTRUCTED AND OF THEIR CONTROL

Loss of ability of the dry yeasts expressed
in percentage of the loss of gassing
power of the original strain

| | rehydration at 20° C. | rehydration at 38° C. |
|---|---|---|
| M5 | 100 | 100 |
| M5 fill | 44 | 26 |
| SP1 | 100 | 100 |
| SP1 fill | 49 | 52 |

These results show that the fil1 gene, i.e. the gene CYR1/CDC35 carrying the fil1 mutation allows the construction of industrial strains which present a loss during drying which is smaller.

b/ Drying of the Strain S47 and of the Strain S47 fil500

The same experiment has been carried out with the strain S47 fil500 obtained from a selection based on freeze resistance during the fermentation phase, taking into account that very often the stress resistances are crossed. The fermentative abilities have been measured according to test A1 as far as the fresh yeast is concerned, and according to test A′1 as far as the dry yeast is concerned with rehydration at 20° C. The results are given in table 5-B.

TABLE 5-B
EVALUATION OF THE LOSS OF GASSING
POWER AFTER DRYING OF THE STRAIN
S47 fil500 AND OF ITS CONTROL

| | Loss of gassing power of the dry |
|---|---|
| | yeasts expressed in percentage |
| | of the loss of gassing power |
| | of the starting strain |
| | rehydration at 20° C. |
| S47 | 100 |
| S47 fil500 | 66 |

This table 5-B confirms that the fil phenotype allows to diminish the loss during drying of very active yeasts and consequently to obtain very active dry yeasts.

c/ Drying of AT25=S47 fil400 and S47 Strains

The above experiment has been repeated several times with AT25=S47 fil400 strain, which, in all of the tests, proved to be the best strain among AT strains series constructed according to the process described in in example 2.

The average results are as follows:

| | | | A'1 test on | Loss due to |
|---|---|---|---|---|
| | N/DM | A1 test on | active dry | drying and |
| | Nitrogen on | yeast before | yeast at 95% | rehydration at |
| | dry matter | drying | dry matters | 38° C. |
| S47 | 8.2% | 152 | 105 | 31% |
| AT25 | 8.3% | 165 | 135 | 18% |

### EXAMPLE 6

### Use of the fil Strains in Frozen Dough

In this example, are described the results during freezing of fil strains obtained in example 2: the strain S47 fil400 (=AT25), the strain S47 fil500 (=AT28) and the strain AT26. The stability during freezing of these fil strains is compared with that of the strain S47, control strain from which they originate. To do this, the loss of CO₂ production associated with a prolonged storage of frozen pieces of dough at −20° C. has been measured.

The yeasts are cultivated on molasses, in fed batch on pilot fermentors in order to obtain active biomasses having a maximum nitrogen content of 8% on dry matter, and then they were used in order to prepare pieces of dough according to the conditions described in the test C1. For each strain, the value of the reference gas (CO₂) production corresponds to the value of the gas production measured after 1 day of conservation at −20° C. The other pieces of dough were thawed after 1 week, 2 weeks, 3 weeks, 4 weeks and 6 weeks; they allow to monitor the change of the gassing power as a function of the duration of conservation at −20° C. Furthermore, the stability in frozen dough pieces of each strain after n days of conservation at −20° C. was measured using the following ratio:

$$\frac{\begin{matrix}{CO}_{2} release according to C1 test on \\ day n of conservation at - 20 ° C .\end{matrix}}{\begin{matrix}{CO}_{2} release according to C1 test after \\ the first day of conservation at - 20 ° C .\end{matrix}}$$

The results are collected in table 6-A (evolution of the fermentative activity C1 test) and in FIG. 6-1 (stability in frozen doughs maintained at −20° C.).

A very clear improvement of the stability during freezing was noticed for the 3 mutants AT25, AT26 and AT28 (FIG. 6-1); under these conditions of test C1, the control strain S47 maintains after 1 month 54% of its reference gassing power, while the mutants conserve, under the same conditions between 80 and 87% of their reference gassing power. Furthermore and due to the fact that the mutations did not lead to an important penalization of the fermentative ability, all the mutants attain quickly (from 8 to 15 days of conservation at −20° C.) levels of fermentative ability higher than those of control S47 (table 6-A).

TABLE 6-A
EVOLUTION OF THE GASSING POWER
AS A FUNCTION OF THE
DURATION OF STORAGE AT-20° C.

| | Fermentative ability after conservation at −20° C. | | | | | |
|---|---|---|---|---|---|---|
| | 1 day | 9 days | 15 days | 23 days | 30 days | 45 days |
| S47 | 115 | 102 | 89 | 77 | 62 | 47 |
| AT25 | 104 | 98 | 92 | 87 | 85 | 65 |
| AT26 | 108 | 105 | 99 | 94 | 90 | 69 |
| AT28 | 105 | 100 | 98 | 95 | 91 | 78 |

These results led to multiply the pilot tests with the 3 strains AT25, AT26 and AT28 in order to:

select the best strain among these 3 strains

check the selected strain did not have any hampering character, in particular in the frame of different bread-making conditions.

The results with the 3 strains AT25, AT26 and AT28 showed that the best strain giving the most regular results after more than 10 trials is the AT25 strain.

In particular, the AT25 strain in test R gives the same result that the S47 strain.

The baker's yeasts obtained with the AT25 strain have been tested in numerous bread-making processes, particularly frozen doughs, realized in numerous different recipes to verify they did not give any bad taste or odour to bread, and this requirement is clearly fulfilled.

The baker's yeasts obtained with this strain lead with frozen doughs to a spectacular decrease in the proof time, that is to say in the fermenting time after thawing necessary to obtain a given volume to the dough.

The essential characteristics of the AT25 strain are as follows:

production yield on molasses 50 about 2% inferior to that of S47 strain

obtaining of compressed fresh yeast giving at 7.6% of nitrogen on dry matters 148 ml in average of CO₂ in test A1 in 2 hours, i.e. a slightly more rapid gassing power (+10%) than that of the S47 compressed fresh baker's yeast strain

preservation after 7 days at 26° C. in polyethylene plastic bags of at least 40% of the initial activity of freshly produced compressed bakers' yeast in test A1

no unpleasant smell, no bad taste given to breads, on the contrary, in the processes including a long storage of the frozen doughs (60 days), breads have pure taste with no yeast taste

better stress resistance to freezing leading in test C1 to a conservation stability defined by the ratio:

$$\frac{\begin{matrix}{CO}_{2} release according to test C1 after a month \\ ( 30 days ) of conservation at - 20 ° C .\end{matrix}}{\begin{matrix}{CO}_{2} release according to test C1 after \\ one day of conservation at - 20 ° C .\end{matrix}} \geq 80 \%$$

### EXAMPLE 7

### Construction of New fil Strains Derived from S47 fil400=AT25

The genetic analysis of the segregants obtained from AT25 strains led to think that the mutations did concern several genes.

The following step was then used in an attempt to obtain more performing strains from AT25.

The AT25 segregants were crossed according to their sign with the laboratory haploids W303-1A of mating type a or α. The polyploid strains obtained were subjected to T7 test after 2 weeks of freezing, and classified in this test in comparison with AT25 and S47 and diploid W303-1A.

In this first series of tests, the following values are obtained for the control strains:

| | | T6 |
|---|---|---|
| | T7 | glucose consumption after |
| | 2 weeks | 90 minutes |
| AT25 | 28% | ≧5.0 |
| S47 | 14% | ≧5.0 |
| W303-1A diploid | 12% | 3.5 |

27 polyploid strains are obtained with a result equal or superior to S47 strain in T7 test, 7 of which show a result superior to AT25 strain. All these strains have a glucose consumption in T6 test higher than that of diploid W303-1A.

The segregants issued from AT25, which after crossing with W303-1A haploid revealed a polyploid strain giving in test T7 a result as least equal to S47 strain, are then crossed between them.

Polyploid strains thus obtained are selected with T6 and T7 tests, the selection criteria being as follows:

glucose consumed within 90 minutes at least equal to 80% of the glucose consumed by AT25 or S47

result in T7 after 1 day and after 3 weeks of freezing superior to the result obtained with AT25.

9 polyploid strains corresponding to this definition were obtained, in a first experiment, 3 of which have been deposited at the C.N.C.M. These 3 strains gave the following results:

| | T6 glucose | T7 after | T7 | | T4 μmax. |
|---|---|---|---|---|---|
| | consumption | 1 day | after | 3 cAMP | on |
| | in | of | weeks | of | molasses |
| Strain | 90 minutes | freezing | freezing | response | 0.5% |
| AT251 | 5.8 | 68 | 38 | + | 0.6 |
| AT252 | 5.8 | 69 | 40 | − | 0.6 |
| AT254 | 5.7 | 66 | 40 | + | 0.6 |
| AT25 | 5.6 | 50 | 26 | ++ | 0.6 |
| S47 | 5.8 | 28 | 6 | ++ | 0.6 |

AT251 strain was deposited at C.N.C.M., 25 rue du Docteur Roux, F-75724 PARIS cedex, under the n° I-2222, in accordance with the Budapest Treaty.
AT252 strain was deposited at C.N.C.M., 25 rue du Docteur Roux, F-75724 PARIS cedex, under the n° I-2223, in accordance with the Budapest Treaty.
AT254 strain was deposited at C.N.C.M., 25 rue du Docteur Roux, F-75724 PARIS cedex, under the n° I-2224, in accordance with the Budapest Treaty.

These results show that the 3 AT251, AT252 and AT254 strains are good candidates to give in frozen doughs better results than with AT25 strain. These results prove that the process of improvement of an industrial mutant preferably with fil type, and obtained according to the process subject of the invention, bearing mutations on several genes compared to the not-mutated industrial strain, consisting:

in crossing the segregants issued from this industrial mutant with a laboratory haploid strain to select the segregants of the said industrial mutant giving to the polyploids obtained with the laboratory strain an improvement in the required property(ies).

in crossing the industrial segregants thus selected one with the other and in selecting the polyploids thus obtained according to the criteria of the fil phenotype allows to obtain improved industrial fil strains.

This process is also one of the subjects of the invention, as well as the strains of the same type or kind as the AT251, AT252 and AT254 strains it allows to obtain.

1. Process for obtaining new eukaryotic strains, preferably new yeast strains, and even more preferentially strains of Saccharomyces cerevisiae conserving stress resistance in the presence of fermentable sugars such as glucose, comprising the following steps:
a classic mutagenic treatment is carried out on the cells of a starting strain,
the cells having undergone the said mutagenic treatment are cultured so as to obtain a stationary phase,
the said cells in stationary phase are incubated in the presence of at least one fermentable sugar selected from the group comprising glucose, maltose, and sucrose, this sugar being present in a quantity such that the cells enter an active metabolic state (fermentation and/or growth) of this sugar,
said cells in active metabolic state are subjected to one or several stresses leading to a mortality rate of at least 99% with respect to the starting population,
the surviving cells are isolated and
those of the surviving cells which respond to the following criteria which characterize the fil phenotype are selected, i.e.:
a growth, evaluated by production or production yield of biomass over sugar in a given time or by a growth rate, under identical culture conditions, at least equal to 80% of the starting strain, and preferably at least equal to 90% of the starting strain,
a CO₂ release, or a metabolite production, in identical conditions, at least equal to 80%, and preferably at least equal to 90% of the starting strain,
a stress resistance, corresponding to a survival rate at least 2 times higher, preferably at least 3 times higher, more preferentially at least 5 times higher, and even more preferentially at least 10 times higher than the survival rate of the starting strain, under identical phase conditions corresponding to a growth or active metabolism followed by a heat shock of at least 20 minutes at 52° C., or at least 1.5 times higher, preferably at least 2 times higher, more preferably at least 3 times higher, and even more preferentially at least 5 times higher than the survival rate of the starting strain, under identical conditions of growth phase followed by freezing for a period of at least 24 hours at −20° C. or at a lower temperature,
maintenance of these properties after repeated cultures on non selective medium, such as YPD medium, so as to verify that the fil phenotype obtained by the mutation is perfectly stable and permanent.

2. Process according to claim 1, wherein it is checked that any useful secondary property has not been lost and that any hampering property has not appeared.

3. Process according to claim 1, wherein the starting strain is an industrial strain.

4. Process according to claim 3, wherein an industrial fil mutant carrying several mutations is obtained and wherein:
the segregants issued from this industrial mutant are crossed with a laboratory haploid strain to select the segregant issued from this industrial mutant giving to the polyploids obtained with the laboratory strain an improvement in the required properties;
the segregants thus selected are crossed one with the other;
the polyploids obtained are selected according to the criteria of fil phenotypes defined in claim 1.

5. Process according to claim 1, wherein the selected fil strains preferably have the property of conserving, in growth and/or fermentation phase on fermentable sugars, at least 50%, preferably at least 60%, more preferentially at least 70%, and even more preferentially at least 80% of their survival rate with respect to the survival rate in stationary phase measured under the same conditions after a heat or freeze shock.

6. Process according to claim 1, wherein the cells obtained after mutagenesis treatment are introduced into pieces of dough subjected to at least 100 cycles of freezing/thawing after a first fermentation of the dough of 30 minutes at 30° C.

7. New industrial eukaryotic strain, preferably of yeast and still more preferably belonging to the Saccharomyces genus having the fil phenotype, obtainable by the process according to claim 1.

8. New industrial yeast strain, preferably belonging to the Saccharomyces genus and still more preferably belonging to the Saccharomyces cerevisiae species having the fil phenotype, obtainable by the process according to claim 2.

9. New strain according to claim 7, belonging to Saccharomyces cerevisiae species.

10. New yeast strain according to claim 7 having a survival rate, in growth phase on fermentable sugars, of at least 50%, preferably at least 60%, more preferably at least 70% and still more preferably at least 75%, after a heat treatment of 20 minutes at 52° C., , the growth phase being defined as a reculturing on fermentable sugar (glucose) of 10 minutes at 30° C. after stationary phase.

11. New yeast strain according to claim 8 having a survival rate, in growth phase on fermentable sugars, of at least 50%, preferably at least 60%, more preferably at least 70% and still more preferably at least 75%, after a heat treatment of 20 minutes at 52° C., , the growth phase being defined as a reculturing on fermentable sugar (glucose) of 10 minutes at 30° C. after stationary phase.

12. New industrial yeast according to claim 7 whose stability to freezing in lumps of dough incubated 60 minutes at 30° C. before freezing and containing 20 g of flour, 15 g of water, 1 g of sucrose, 0.405 g of NaCl, 0.06 g of (NH₄)₂SO₄ and 160 mg of dry matter of the considered strain, defined by the ratio between the release of CO₂ at 30° C. after 1 month or 30 days of conservation at −20° C. and the release of CO₂ at 30° C. after 1 day of conservation at −20° C., is at least equal to 80%, preferably at least equal to 85% and more preferably at least equal to 90%.

13. New industrial yeast strain according to claim 8, whose stability to freezing in lumps of dough incubated 30 minutes at 30° C. before freezing and containing 20 g of flour, 15 g of water, 0.405 g of NaCl, 0.06 g of (NH₄)₂SO₄ and 160 mg of dry matter of the considered strain, measured by the ratio between the release of CO₂ at 30° C. after 1 month or 30 days of conservation at −20° C. and the release of CO₂ at 30° C. after 1 day of conservation at −20° C., is at least higher than 80%, preferably at lest higher than 85% and more preferably at least higher than 90%.

14. New yeast strain according to claim 7, whose loss of released gas after drying of the biomass harvested in a phase close to exponential growth phase is at most equal to 67%, preferably at most equal to 50% of the loss of released gas after drying of yeasts obtained using, the corresponding starting strain or a control strain having the same characteristics.

15. New strain PVD1150=M5 fil1 deposited at C.N.C.M. under the n° I−2031 and the n° I-2203.

16. New strain KL1=W303 fil2 deposited at C.N.C.M. under the n° I−2032.

17. New strain FD51=HL816 fil300 deposited at C.N.C.M. under the n° I-2033.

18. New strain FDH16-22=HL822 fil300 deposited at C.N.C.M. under the n° I−2034.

19. New strain AT25=S47 fil400 deposited at C.N.C.M. under the n° I−2035.

20. New strain AT28=S47 fil500 deposited at C.N.C.M. under the n° I−2036.

21. New strain AT251 deposited at C.N.C.M. under the n° I-2222.

22. New strain AT252 deposited at C.N.C.M. under the n° I-2223.

23. New strain AT254 deposited at C.N.C.M. under the n° I-2224.

24. New strains belonging to the same kind than strains AT25 and AT28.

25. New strains belonging to the same kind than strains AT251, AT252 and AT24.

26. Mutant gene obtainable by isolation from one of the mutant strains obtainable by the process according to claim 1.

27. Gene according to claim 26, conferring the fil phenotype to one of the strains according to claim 7.

28. Gene CDC35=CYR1 carrying a mutation conferring the fil phenotype.

29. Gene according to claim 28, wherein the mutation is a change of a G base into an A base in the region of the gene CDC35/CYR1 coding for the catalytic site of the enzyme, equivalent to a change of an acidic amino acid (glutamic acid) into a basic amino acid (lysine) at position 1682 of the protein.

30. Gene GPR1 carrying a mutation conferring the fil phenotype.

31. Gene according to claim 30 carrying the mutation of the KL1=W303 fil 2 strain.

32. Gene having properties similar or equivalent to those of one of the genes according to claim 27, i.e. gene carrying a mutation conferring the fil phenotype, and belonging to the family of genes:
coding for proteins having a function comparable to that of a protein coded by one of the genes according to claim 27 in yeast or another eukaryote,
coding for proteins associated with the protein coded by one of the genes according to claim 27,
coding for proteins having similar sequences, i.e. at least 60% homology, preferably at least 70% homology and still more preferably at least 80% homology with the protein coded by one of the genes according to claim 27.

33. Gene according to claim 32, coding for a protein associated with the protein coded by the GPR1 gene according to claim 30 wherein the said gene may be a GPA gene as the GPA2 gene of yeast carrying a mutation which confers the fil phenotype.

34. Eukaryotic strain transformed in a manner so that at least certain of the alleles of the gene according to claim 26 or genes analogous to these genes carry a mutation which confers the fil phenotype.

35. Yeast strain transformed in a manner so that at least certain of the alleles of the gene according to claim 26 carry a mutation which confers the fil phenotype.

36. Process for obtaining baker's yeast intended for frozen doughs comprising the use of a strain according to claim 7.

37. Process for obtaining baker's yeast intended for frozen doughs comprising the use of a strain according to claim 8.

38. Process for obtaining dry baker's yeast comprising the use of a strain according to claim 7.

39. Process for obtaining brewery yeast comprising the use of a strain according to claim 8.

40. Process for obtaining brewery yeast comprising the use of a strain according to claim 7.

41. Process for obtaining yeasts intended for the production of alcohol comprising the use of a strain according to claim 7.
